SEQUENCE LISTING (13 sequences)

SEQ ID NO: 1 - protein, 471 aa, synthetic construct
  source 1..471: note = PAAP Protein
MSPSGVRNVIILIGDGMGFSQLQLTKLVYGHLNMEDFPYTGIELTDSLSGEVTDSAAAGTAIATGVKTYNRMISTTNVTGKLVNLTTLLEIAQMLGKATGLVTTTRITHATPAVFASHVPDRDMEEEIARQLILHNVTVLMGGGREKFSEEVLKLAEDYGYSIVYTREDLEKVKDGKVLGLFAEGHLPYVLDRSEEDVSLLEMTKKAIEILEKNPNGFFLMIEGGRIDHACHANDVASIVAETKEFDDVVGYVLDYARRRGDTLVIVLADHETGGLGIGLNYGHSVDIDSIRRIDASIEEMSKEIKSGGDIRDVIRRHTGLELTDEEVKEIEEAKNSTNKYALGNIIGEIISKKLGVGFVSHKHTGEPVPLLAYGPGAENFVGFKHHVDTAKVIAKLMIFGDRSISFTIKGVSKIKGDVTGDYRVDERDAYATLMLLLGDLVDTELENIADMDNNGIIDLLDVMAILQASS

SEQ ID NO: 2 - protein, 207 aa, synthetic construct
  source 1..207: note = MjaOGG Protein
MMLIKKIEELKNSEIKDIIDKRIQEFKSFKNKSNEEWFKELCFCILTANFTAEGGIRIQKEIGDGFLTLPREELEEKLKNLGHRFYRKRAEYIVLARRFKNIKDIVESFENEKVAREFLVRNIKGIGYKEASHFLRNVGYDDVAIIDRHILRELYENNYIDEIPKTLSRRKYLEIENILRDIGEEVNLKLSELDLYIWYLRTGKVLK

SEQ ID NO: 3 - DNA, 29 bp, synthetic construct
  source 1..29: mol_type = other DNA; note = P5 sequence
aatgatacggcgaccaccgagatctacac

SEQ ID NO: 4 - DNA, 24 bp, synthetic construct
  source 1..24: mol_type = other DNA; note = P7 sequence
caagcagaagacggcatacgagat

SEQ ID NO: 5 - DNA, 29 bp, synthetic construct
  source 1..29: mol_type = other DNA; note = P5 prime sequence
gtgtagatctcggtggtcgccgtatcatt

SEQ ID NO: 6 - DNA, 24 bp, synthetic construct
  source 1..24: mol_type = other DNA; note = P7 prime sequence
atctcgtatgccgtcttctgcttg

SEQ ID NO: 7 - DNA, 35 bp, synthetic construct
  source 1..35: mol_type = other DNA; note = Substrate
  modified_base 35: mod_base = OTHER; note = thymidine 3 prime-monophosphate
tctagagcccgcctaatcagcgggcttttttttat

SEQ ID NO: 8 - DNA, 35 bp, synthetic construct; identical to SEQ ID NO: 7
  source 1..35: mol_type = other DNA; note = Product

SEQ ID NO: 9 - DNA, 59 bp, synthetic construct
  source 1..59: mol_type = other DNA; note = Oligonucleotide Substrate
  modified_base 32: mod_base = OTHER; note = 8-Oxo-2-deoxyguanosine
ttttttttttcaagcagaagacggcatacganataatttagaccctaatatcacatcat

SEQ ID NO: 10 - DNA, 31 bp, synthetic construct
  source 1..31: mol_type = other DNA; note = 5 Prime Product
ttttttttttcaagcagaagacggcatacga

SEQ ID NO: 11 - DNA, 27 bp, synthetic construct
  source 1..27: mol_type = other DNA; note = 3 Prime Product
ataatttagaccctaatatcacatcat

SEQ ID NO: 12 - protein, 251 aa, Pyrococcus abyssi
MGSSHHHHHHSSGLVPRGSHMRIVAADTGGAVLDESLQPIGLISTVAVLVEKPYNTAKEFLVKFSDPYNYDLSGRQAILDEINLAIELAKRTKPDVVHLDSTLGGIEVRKLDEPTIEALSISDKGKEVWKDLSKDVQPLAKKFWEETGIEILAIGKSSVPVRIAEIYSGIFSVKWAIEEVKKGHKDHLIIGLPRYMGVEIRDGKITGRTLDPREGNLYGEISIEVPNNIKWEVYPNPLVRRFHIFEIWGEY

SEQ ID NO: 13 - protein, 249 aa, Pyrococcus furiosus
MGSSHHHHHHSSGLVPRGSHMRIVAADTGGAVLDETFEPIGLIATVAVLVEKPYRSAKEVMVKYANPYDYDLTGRQAIRDEVLLAIELARKVKPDVIHLDSTLGGIELRKLDEPTIDALGISDKGKEVWKELSKDLQPLARKFWEETNIEIVAIGKSSVPVRIAEIYAGIYSAKWGIENVEKEGHLIIGLPRYMEVNIKDGKIIGRSLDPREGGLYGSAEVSVPEGVKWEIYPNPVARRFMIFEIFSKR

(19) United States
(12) Patent Application Publication
(10) Pub. No.: US 2024/0102067 A1
(43) Pub. Date: Mar. 28, 2024

(54) Resynthesis Kits and Methods

(71) Applicant: Illumina, Inc., San Diego, CA (US)

(72) Inventors: Kay Klausing, San Diego, CA (US); Jonathan Boutell, Bishops Stortford (GB); Trina Osothprarop, San Diego, CA (US); Oliver Miller, Linton (GB); Justin Robbins, San Diego, CA (US)

(73) Assignee: Illumina, Inc., San Diego, CA (US)

(21) Appl. No.: 18/473,971

(22) Filed: Sep. 25, 2023

Related U.S. Application Data
(60) Provisional application No. 63/410,162, filed on Sep. 26, 2022.

(51) Int. Cl. C12P 19/34; C12Q 1/6874

(52) U.S. Cl. CPC C12P 19/34; C12Q 1/6874

(57) ABSTRACT

This disclosure relates to novel mjresynthesis kits and methods, in particular for use in pairwise sequencing.

### CROSS-REFERENCE TO RELATED APPLICATION

This application claims the benefit of U.S. Provisional Patent Application No. 63/410,162, filed Sep. 26, 2022 and entitled “Resynthesis Kits and Methods,” the disclosure of which is hereby incorporated by reference in its entirety.

### FIELD

This disclosure relates to novel resynthesis kits and methods, in particular for use in pairwise sequencing.

### SEQUENCE LISTING

The instant application contains a Sequence Listing which has been submitted electronically in xml format and is hereby incorporated by reference in its entirety. Said xml copy was created on Sep. 20, 2023, is named 85491_08200_US.xml, and is 16.1 kilobytes in size.

### BACKGROUND

The detection of analytes such as nucleic acid sequences that are present in a biological sample has been used as a method for identifying and classifying microorganisms, diagnosing infectious diseases, detecting and characterising genetic abnormalities, identifying genetic changes associated with cancer, studying genetic susceptibility to disease, and measuring response to various types of treatment. A common technique for detecting analytes such as nucleic acid sequences in a biological sample is nucleic acid sequencing.

Advances in the study of biological molecules have been led, in part, by improvement in technologies used to characterise the molecules or their biological reactions. In particular, the study of the nucleic acids DNA and RNA has benefited from developing technologies used for sequence analysis.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing pairwise sequencing. This involves sequencing one strand of the template (a first sequencing read, “read 1”), synthesising the complementary version of the template (resynthesis), and then sequencing the complementary version of the template (a second sequencing read, “read 2”).

Whilst methods exist for synthesising the complementary version of the template, there remains a need to develop new kits and methods for such resynthesis.

### SUMMARY

According to an aspect of the present disclosure, there is provided a resynthesis kit comprising a thermophilic phosphatase and a polymerase.

Preferably, the kit comprises the thermophilic phosphatase at a concentration of about 0.01 μM to about 1000 μM, about 0.02 μM to about 100 μM, about 0.05 μM to about 50 μM, about 0.1 μM to about 20 μM, or about 0.2 μM to about 10 μM.

Preferably, the thermophilic phosphatase is derived from a thermophile, wherein the thermophile is of the genus Pyrococcus.

Preferably, the thermophilic phosphatase comprises an amino acid sequence as defined in SEQ ID NO: 1, or a functional variant or functional fragment thereof.

Preferably, the thermophilic phosphatase has a denaturation temperature of above 40° C., above 45° C., above 50° C., above 55° C., above 60° C., above 65° C., above 70° C., above 75° C., above 80° C., above 85° C., above 90° C., above 95° C., above 100° C., above 105° C., or above 110° C.

Preferably, the kit comprises the polymerase at a concentration of about 0.01 μM to about 1000 μM, about 0.02 μM to about 100 μM, about 0.05 μM to about 50 μM, about 0.1 μM to about 20 μM, or about 0.2 μM to about 10 μM.

Preferably, the polymerase is a thermophilic polymerase.

Preferably, the thermophilic polymerase has a denaturation temperature of above 40° C., above 45° C., above 50° C., above 55° C., above 60° C., above 65° C., above 70° C., above 75° C., above 80° C., above 85° C., above 90° C., above 95° C., above 100° C., above 105° C., or above 110° C.

Preferably, the kit further comprises a thermophilic glycosylase, preferably a thermophilic oxoguanine glycosylase.

Preferably, the kit comprises the thermophilic glycosylase at a concentration of about 0.01 μM to about 1000 μM, about 0.02 μM to about 100 μM, about 0.05 μM to about 50 μM, about 0.1 μM to about 20 μM, or about 0.2 μM to about 10 μM.

Preferably, the thermophilic glycosylase is derived from a thermophile, wherein the thermophile is of the genus Methanocaldococcus (Methanococcus).

Preferably, the thermophilic glycosylase comprises an amino acid sequence as defined in SEQ ID NO: 2, or a functional variant or functional fragment thereof.

Preferably, the thermophilic glycosylase has a denaturation temperature of above 40° C., above 45° C., above 50° C., above 55° C., above 60° C., above 65° C., above 70° C., above 75° C., above 80° C., above 85° C., above 90° C., or above 95° C.

Preferably, the kit comprises an exonuclease, preferably a thermophilic exonuclease, preferably a thermophilic exonuclease. Preferably, the kit comprises a thermophilic exonuclease at a concentration of between about 1 ug/ml and 1000 ug/ml. Preferably, the thermophilic exonuclease is derived from Pyrococcus abyssi or Pyrococcus furiosus. Preferably, the thermophilic exonuclease comprises an amino acid sequence as defined in SEQ ID NO: 12, or a functional variant or functional fragment thereof. Preferably, the thermophilic exonuclease comprises an amino acid sequence as defined in SEQ ID NO: 13, or a functional variant or functional fragment thereof.

Preferably, the thermophilic phosphatase is in a lyophilized formulation. Preferably, the lyophilized formulation comprises the thermophilic phosphatase, a salt, an exonuclease, a detergent, and any one or more of magnesium chloride, acetate, or sulfate. Preferably, the phosphatase comprises Pyrococcus abyssi alkaline phosphatase.

Preferably, the thermophilic oxoguanine glycosylase is in a lyophilized formulation. Preferably, the lyophilized formulation comprises the thermophilic oxoguanine glycosylase, a salt, and trehalose.

Preferably, the kit comprises at least one selected from the group consisting of: a recombinase, a single-stranded nucleotide binding protein, nucleotide triphosphates (NTPs), an ATP-generating substrate and an ATP-generating enzyme.

Preferably, the kit comprises a recombinase, a single-stranded nucleotide binding protein, nucleotide triphosphates (NTPs), an ATP-generating substrate and an ATP-generating enzyme.

Preferably, the kit further comprises a metal cofactor composition, preferably wherein the metal cofactor composition comprises magnesium ions.

Preferably, the kit further comprises instructions for use of the kit in resynthesis of a nucleic acid template, or pairwise sequencing of a nucleic acid template.

According to a further aspect of the present disclosure, there is provided a resynthesis kit comprising a thermophilic glycosylase and a polymerase. Preferably, the thermophilic glycosylase is as described herein. Preferably, the polymerase is as described herein.

According to a further aspect of the present disclosure, there is provided a use of a resynthesis kit according to any one of claims 1 to 17, in resynthesis of a nucleic acid template, or pairwise sequencing of a nucleic acid sequence.

According to a further aspect of the present disclosure, there is provided a method of conducting resynthesis of a nucleic acid sequence, wherein the method comprises:
removing a blocking phosphate group from a blocked primer using a thermophilic phosphatase to form a deblocked primer; and
forming a second nucleic acid template extending from the deblocked primer using a polymerase and a first nucleic acid template.

Preferably, the thermophilic phosphatase is as defined herein.

Preferably, the polymerase is as defined herein.

Preferably, the blocked primer and/or deblocked primer is immobilised on a solid support, preferably wherein the solid support is a flow cell.

Preferably, the step of forming the second nucleic acid template extending from the deblocked primer is conducted using bridge amplification.

Preferably, the method further comprises a step of detaching the first nucleic acid template by using a thermophilic glycosylase.

Preferably, the thermophilic glycosylase is as defined herein.

Preferably, the method is conducted isothermally.

Preferably, the method is conducted at a temperature of about 50° C. to about 75° C., about 55° C. to about 70° C., or about 60° C. to about 65° C.

Preferably, the method comprises using a resynthesis kit as recited herein.

According to a further aspect of the present disclosure, there is provided a method of sequencing a nucleic acid sequence by pairwise sequencing, wherein the method comprises:
sequencing a first nucleic acid template;
conducting resynthesis of a nucleic acid sequence using a method as recited herein to form a second nucleic acid template; and
sequencing the second nucleic acid template.

Preferably, the step of sequencing the first nucleic acid template and/or the step of sequencing the second nucleic acid template is conducted using a sequencing-by-synthesis technique or a sequencing-by-ligation technique.

Preferably, the method comprises using a resynthesis kit as recited herein.

### DESCRIPTION OF THE DRAWINGS

FIG. 1 shows characteristics of Pyrococcus abyssi phosphatase. The primary amino acid sequence is shown along with the predicted molecular weight, without the hexa-His purification tag, which contributes ˜2 kDa.

FIG. 2 shows in vitro activity of Pyrococcus abyssi phosphatase.

FIG. 3 shows the range of activity of Pyrococcus abyssi phosphatase versus temperature.

FIG. 4 shows in vitro activity assay testing for ambient shipping requirements.

FIGS. 5A-5B show activity testing for Pyrococcus abyssi phosphatase in response to heat shock. (FIG. 5A) No detectable decrease in PAAP activity when incubated at 80° C. for up to 70 min using the in vitro activity assay. (FIG. 5B) Corresponding time points at 20 min and 70 min were assayed for nuclease activity. For the nuclease activity assay, samples were incubated at 1 mg/mL PAAP concentration with pUC19 DNA at 37° C. for 18 hrs. Samples were then run on a 1% Agarose gel stained with Sybr gold to detect the presence of DNA. Presence of nuclease (+control) appears as bands or smears below the “DNA only” band. “E963” is a negative control of a nuclease free protein prep that is also incubated with pUC19. Increasing the heat shock time at 80° C. decreases nuclease activity. “Abyssi NHis” is the PAAP protein.

FIG. 6 shows characteristics and purification of recombinant MjaOGG protein. The primary amino acid sequence is shown along with the predicted molecular weight, without the hexa-His purification tag, which contributes ˜2 kDa.

FIGS. 7A-7B show in vitro activity of the MjaOGG protein, showing activity on single-stranded nucleic acid substrates with an 8-oxo-2-deoxyguanosine modification.

FIG. 8 shows in vitro activity assessed with 8-oxo-2-deoxyguanosine activity assay over different concentrations of MjaOGG protein.

FIG. 9 shows in vitro activity assay testing for ambient shipping requirements. MjaOGG enzyme retains >80% activity when thermal stressed at 55° C. for 10 and >70% activity when thermal stressed at 37° C. for 19 days when assessed in vitro with the 8-oxo-2-deoxyguanosine activity assay. The results of these stress conditions indicate the MjaOGG protein meets the ambient ship requirements.

FIG. 10 shows activity testing for MjaOGG protein in response to heat shock. No detectable nuclease activity when MjaOGG was incubated at 80° C. for 70 min followed by the purification and storage; see lanes 4 and 8. MjaOGG and respective control proteins were incubated with 1 μg of pUC19 DNA lanes 1-4 or 1 μg of lambda H3 DNA lanes 5-8 16 hrs at 37° C. Samples were run on 1% Agarose gel stained with ethidium bromide to detect the DNA. Presence of nuclease lanes 3 and 7 appear as bands or smears below the DNA only band lanes 1 and 5. E963 is a negative control of a nuclease free protein preparation lanes 2 and 6.

FIG. 11 shows the thermal profile of mesophilic and thermophilic enzymes; grey—current mesophilic enzymes utilised on sequencing platforms; white—thermophilic enzymes of the present disclosure.

FIG. 12 shows a comparison between mesophilic enzymes (blue and green) and thermophilic enzymes (red) when used in resynthesis; blue—original MiniSeq recipe using mesophilic enzymes; green—adapted MiniSeq recipe for 2×250 runs but using mesophilic enzymes; red—adapted MiniSeq recipe but using thermophilic enzymes.

FIGS. 13A and 13B show a comparison of E. coli exonuclease and thermostable exonuclease during ambient shipping conditions.

FIG. 14 shows a comparison between the activity of thermostable exonuclease and exonuclease derived from E. coli, when the enzymes are used in sequencing reactions. TEX refers to thermostable exonuclease. Novaseq Exo refers to exonuclease derived from E. coli.

FIG. 15 shows a comparison of activity after staging between thermostable exonuclease in liquid and lyophilized formats. TEX refers to thermostable exonuclease.

FIG. 16 shows a comparison of degradation rates of Pyrococcus abyssi alkaline phosphatase (PAAP) in liquid and lyophilized formats. Lyo form refers to lyophilized form.

FIG. 17A provides data showing activity and stability of oxoguanine glycosylase (OGG) in various excipient formulations used for lyophilization. FIGS. 17B and 17C show activity and stability of oxoguanine glycosylase (OGG) in various lyophilized formats.

FIG. 18 provides data showing sequencing metrics using Pyrococcus abyssi alkaline phosphatase (PAAP) and oxoguanine glycosylase (OGG) in various lyophilized formats.

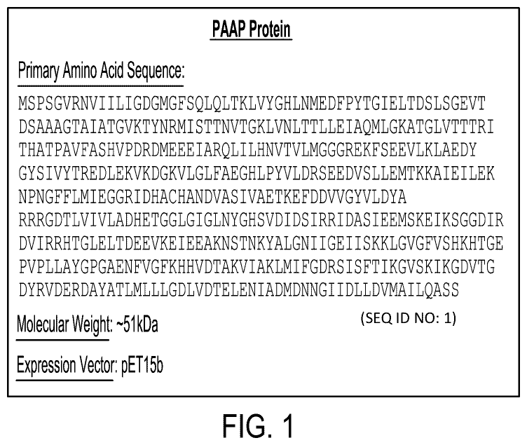

### DETAILED DESCRIPTION

The following described features apply to all aspects and embodiments of the disclosure.

The present disclosure is directed to resynthesis kits and methods.

The present disclosure can be used in sequencing, in particular pairwise sequencing. Methodology applicable to the present disclosure has been described in WO 08/041002, WO 07/052006, WO 98/44151, WO 00/18957, WO 02/06456, WO 07/107710, WO 05/068656, U.S. Ser. No. 13/661,524 and US 2012/0316086, the contents of which are herein incorporated by reference. Further information can be found in US 20060024681, US 200602926U, WO 06110855, WO 06135342, WO 03074734, WO07010252, WO 07091077, WO 00179553 and WO 98/44152, the contents of which are herein incorporated by reference.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts”. Alternatively “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adaptor sequences.

As used herein an “adapter” sequence comprises a short sequence-specific oligonucleotide that is ligated to the 5′ and 3′ ends of each DNA (or RNA) fragment in a sequencing library as part of library preparation. The adaptor sequence may further comprise non-peptide linkers.

As will be understood by the skilled person, a double-stranded nucleic acid will

typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

In one embodiment, the template comprises, in the 5′ to 3′ direction, a first primer-binding sequence (e.g. P5, for example, comprising the sequence as defined in SEQ ID NO: 3), an index sequence (e.g. i5), a first sequencing binding site (e.g. SBS3), an insert, a second sequencing binding site (e.g. SBS12), a second index sequence (e.g. i7) and a second primer-binding sequence (e.g. P7′, for example, comprising the sequence as defined in SEQ ID NO: 6). In another embodiment, the template comprises, in the 3′ to 5′ direction, a first primer-binding site (e.g. P5′, which is complementary to P5, for example, comprising the sequence as defined in SEQ ID NO: 5), an index sequence (e.g. i5′, which is complementary to I5), a first sequencing binding site (e.g. SBS3′ which is complementary to SBS3), an insert, a second sequencing binding site (e.g. SBS12′, which is complementary to SBS12), a second index sequence (e.g. i7′, which is complementary to 17) and a second primer-binding sequence (e.g. P7, which is complementary to P7′, for example, comprising the sequence as defined in SEQ ID NO: 4). Either template is referred to herein as a “template strand” or “a single stranded template”. Both template strands annealed together is referred to herein as “a double stranded template”.

A sequence comprising at least a primer-binding sequence (preferably a combination of a primer-binding sequence, an index sequence and a sequencing binding site) may be referred to herein as an adaptor sequence, and a single insert is flanked by a 5′ adaptor sequence and a 3′ adaptor sequence. The first primer-binding sequence may also comprising a sequencing primer for the index read (I5). “Primer-binding sequences” may also be referred to as “clustering sequences” in the present disclosure, and such terms may be used interchangeably.

The P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although, in embodiments, the disclosure is not limited to sequences of this length. The precise identity of the amplification primers (e.g. lawn primers), and hence the cognate sequences in the adaptors, are generally not material to the disclosure, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05068656, whose contents are incorporated herein by reference in their entirety. The tag can be read at the end of the first read, or equally at the end of the second read, for example using a sequencing primer complementary to the strand marked P7. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S.60/899,221. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation is used.

Following denaturation, a single-stranded template library can be contacted in free solution onto a solid support comprising surface capture moieties (for example P5 and P7 lawn primers). This solid support is typically a flowcell, although in alternative embodiments, seeding and clustering can be conducted off-flowcell using other types of solid support.

By way of brief example, following attachment of the P5 and P7 primers to the solid support, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) lead to the formation of (monoclonal) clusters or colonies of template molecules bound to the solid support.

Thus, solid-phase amplification by either the method analogous to that of WO 98/44151 or that of WO 00/18957 (the contents of which are incorporated herein in their entirety by reference) will result in production of a clustered array comprised of colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582. Further information on amplification can be found in WO0206456 and WO07107710, the contents of which are incorporated herein in their entirety by reference. Through such approaches, a cluster of single template molecules is formed.

To facilitate sequencing, it is preferable if one of the strands is removed from the surface to allow efficient hybridisation of a sequencing primer to the remaining immobilised strand. Suitable methods for linearisation are described in more detail in application number WO07010251, the contents of which are incorporated herein by reference in their entirety.

Sequence data can be obtained from both ends of a template duplex by obtaining a sequence read from one strand of the template from a primer in solution, copying the strand using immobilised primers, releasing the first strand and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′ -end. If the first strand is removed from the surface, the sequence of the second strand can be read. This gives a sequence read from both ends of the original fragment.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB/2007/001770, the contents of which are incorporated herein by reference in their entirety. Alternatively, a separate reaction may be carried out containing each of the modified nucleotides added individually.

The modified nucleotides may carry a label to facilitate their detection. In a particular embodiment, the label is a fluorescent label. Each nucleotide type may carry a different fluorescent label. However the detectable label need not be a fluorescent label. Any label can be used which allows the detection of the incorporation of the nucleotide into the DNA sequence. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

Alternative methods of sequencing include sequencing by ligation, for example as described in U.S. Pat. No. 6,306,597 or WO06084132, the contents of which are incorporated herein by reference.

Sequencing may involve pairwise sequencing. The typical steps of pairwise sequencing are known and have been described in WO 2008/041002, the contents of which are herein incorporated by reference. However, the key steps will be briefly described.

A typical starting point is a plurality of single stranded templates which are attached to the same surface as a plurality of immobilised primers that are complementary to the 3′ end of the immobilised template. The immobilised primers may be reversibly blocked to prevent extension. The single stranded templates may be sequenced using a hybridised primer at the 3′ end. The sequencing primer may be removed after sequencing, and the immobilised primers deblocked to release an extendable 3′ hydroxyl. These immobilised primers may be used to copy the template using bridged strand resynthesis to produce a second immobilised template that is complementary to the first. Removal of the first template from the surface allows the newly single stranded second template to be sequenced, again from the 3′ end. Thus, both ends of the original immobilised template can be sequenced. Such a technique allows paired end reads where the templates are amplified using a single extendable immobilised primer, for example as described in Polony technology (Nucleic Acids Research 27, 24, e34(1999)) or emulsion PCR (Science 309, 5741, 1728-1732 (2005); Nature 437, 376-380 (2005)).

In a typical process for conducting pairwise sequencing, the first immobilised template is covalently attached to the surface via a first immobilised primer (e.g. P5 or P7 primers). The surface also comprises a second immobilised primer (e.g. P7 or P5 primers) which is blocked at the 3′ end, preventing amplification and/or polymerisation. Accordingly, once the first immobilised template has been sequenced (the first sequencing read, “read 1”), the second immobilised primer may need to be deblocked to allow bridge amplification to proceed for generation of the second immobilised template. Typically, a phosphate group at the 3′ end of the second immobilised primer is used for blocking. In previous methods, deblocking has typically been conducted using a mesophilic phosphatase, which removes a phosphate blocking group from a 3′ end of the second immobilised primer.

Bridge amplification then allows synthesis of the second immobilised template, which is complementary to the first immobilised template. This process is termed resynthesis.

A further step of linearisation allows the first immobilised template to be detached and washed away from the surface, by cleaving a covalent bond between the first immobilised template and the first immobilised primer. In previous methods, this has typically been conducted using a mesophilic glycosylase. This then leaves the second immobilised template available for sequencing (the second sequencing read, “read 2”).

The present disclosure has identified that changing the type of phosphatase used (i.e. from a mesophilic phosphatase to a thermophilic phosphatase) during resynthesis provides improved sequencing metrics, in particular reduced error rates during the second sequencing read. A further advantage includes reductions in overall run time.

In an embodiment, the present disclosure is directed to a resynthesis kit comprising a thermophilic phosphatase and a polymerase.

As used herein, the term “thermophilic” or “thermostable” may refer to a protein that does not substantially denature at high temperature, for example above 40° C., above 45° C., above 50° C., above 55° C., above 60° C., above 65° C., above 70° C., above 75° C., above 80° C., above 85° C., above 90° C., above 95° C., above 100° C., above 105° C., or above 110° C.

As used herein, the term “phosphatase” may refer to an enzyme which catalyses the following reaction:

(X)ₙ(3′)O—Pᵢ→(X)ₙ-(3′)O—H+Pᵢ

wherein X refers to a nucleotide (e.g. a nucleotide comprising a nitrogen-containing base such as cytosine, guanine, adenosine, thymine or uracil), “n” refers to the total number of nucleotides in the (poly)nucleotide chain, (3′) O refers to an oxygen atom at the 3′ end of the (poly)nucleotide chain, and Pᵢ refers to a phosphate residue.

The resynthesis kit may comprise the thermophilic phosphatase at a concentration of about 0.01 μM to about 1000 μM, about 0.02 μM to about 100 μM, about 0.05 μM to about 50 μM, about 0.1 μM to about 20 μM, or about 0.2 μM to about 10 μM.

The thermophilic phosphatase may preferably be derived from a thermophile, wherein the thermophile is of the genus Pyrococcus. In one embodiment, the thermophile may be Pyrococcus abyssi.

In a preferred embodiment, the thermophilic phosphatase may comprise the following sequence, or a functional variant or functional fragment thereof:

(SEQ ID NO: 1)
MSPSGVRNVIILIGDGMGFSQLQLTKLVYGHLNMEDFPYTGIELTDSLS
GEVTDSAAAGTAIATGVKTYNRMISTTNVTGKLVNLTTLLEIAQMLGKA
TGLVTTTRITHATPAVFASHVPDRDMEEEIARQLILHNVTVLMGGGREK
FSEEVLKLAEDYGYSIVYTREDLEKVKDGKVLGLFAEGHLPYVLDRSEE
DVSLLEMTKKAIEILEKNPNGFFLMIEGGRIDHACHANDVASIVAETKE
FDDVVGYVLDYARRRGDTLVIVLADHETGGLGIGLNYGHSVDIDSIRRI
DASIEEMSKEIKSGGDIRDVIRRHTGLELTDEEVKEIEEAKNSTNKYAL
GNIIGEIISKKLGVGFVSHKHTGEPVPLLAYGPGAENFVGFKHHVDTAK
VIAKLMIFGDRSISFTIKGVSKIKGDVTGDYRVDERDAYATLMLLLGDL
VDTELENIADMDNNGIIDLLDVMAILQASS

The thermophilic phosphatase may preferably have a denaturation temperature of above 40° C., above 45° C., above 50° C., above 55° C., above 60° C., above 65° C., above 70° C., above 75° C., above 80° C., above 85° C., above 90° C., above 95° C., above 100° C., above 105° C., or above 110° C. More preferably, the thermophilic phosphatase may have a denaturation temperature of between about 40° C. to about 200° C., about 45° C. to about 195° C., about 45° C. to about 190° C., about 55° C. to about 185° C., about 60° C. to about 180° C., about 65° C. to about 175° C., about 70° C. to about 170° C., about 75° C. to about 165° C., about 80° C. to about 160° C., about 85° C. to about 155° C., about 90° C. to about 150° C., about 95° C. to about 145° C., or about 100° C. to about 140° C.

The resynthesis kit further comprises a polymerase. Preferably, the polymerase may be a thermophilic polymerase. In some preferred embodiments, the polymerase may be a DNA polymerase. In other preferred embodiments, the polymerase may be a RNA polymerase. The polymerase may be provided separately from the thermophilic phosphatase. For example, the polymerase may be in a different container to the thermophilic phosphatase.

As used herein, the term “polymerase” may refer to an enzyme that produces a complementary replicate of a nucleic acid molecule using the nucleic acid as a template strand. Typically, DNA polymerases bind to the template strand and then move down the template strand sequentially adding nucleotides to the free hydroxyl group at the 3′ end of a growing strand of nucleic acid. DNA polymerases typically synthesise complementary DNA molecules from DNA templates and RNA polymerases typically synthesise RNA molecules from DNA templates (transcription). Polymerases can use a short RNA or DNA strand, called a primer, to begin strand growth. Some polymerases can displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

The resynthesis kit may comprise the polymerase at a concentration of about 0.01 μM to about 1000 μM, about 0.02 μM to about 100 μM, about 0.05 μM to about 50 μM, about 0.1 μM to about 20 μM, or about 0.2 μM to about 10 μM.

The polymerase may preferably have a denaturation temperature of above 40° C., above 45° C., above 50° C., above 55° C., above 60° C., above 65° C., above 70° C., above 75° C., above 80° C., above 85° C., above 90° C., above 95° C., above 100° C., above 105° C., or above 110° C. More preferably, the polymerase may have a denaturation temperature of between about 40° C. to about 200° C., about 45° C. to about 195° C., about 45° C. to about 190° C., about 55° C. to about 185° C., about 60° C. to about 180° C., about 65° C. to about 175° C., about 70° C. to about 170° C., about 75° C. to about 165° C., about 80° C. to about 160° C., about 85° C. to about 155° C., about 90° C. to about 150° C., about 95° C. to about 145° C., or about 100° C. to about 140° C.

Preferably, the resynthesis kit may further comprise a thermophilic glycosylase. More preferably, the thermophilic glycosylase is a thermophilic oxoguanine glycosylase. The thermophilic glycosylase may be provided separately from the thermophilic phosphatase and/or the polymerase. For example, the polymerase may be in a different container to the thermophilic phosphatase and/or the polymerase.

As used herein, the term “glycosylase” may refer to an enzyme which catalyses the removal of a nitrogenous base from one of the nucleotides in a (poly)nucleotide chain by breaking a N-glycosidic bond, resulting in the formation of an apurinic/apyrimidinic site (AP site). For DNA chains, the glycosylase may recognise any nitrogenous base (e.g. purine or pyrimidine) which is not selected from cytosine (C), guanine (G), adenine (A) and thymine (T); for RNA chains, the glycosylase may recognise any nitrogenous base (e.g. purine or pyrimidine) which is not selected from cytosine (C), guanine (G), adenine (A) and uracil (U). Examples of typical nitrogenous bases recognised by glycosylases include oxoguanine (e.g. 8-oxoguanine) and alkylpurines.

Glycosylases may be monofunctional, such that they only possess glycosylase activity (i.e. breaking of the N-glycosidic bond)—cleavage of a phosphodiester bond in the sugar-phosphate backbone may then occur in an uncatalysed manner by elimination. Other glycosylases may be bifunctional, such that they also possess AP lyase activity by catalysing the phosphodiester bond of the (poly)nucleotide chain. Preferably, the glycosylase is bifunctional (i.e. possesses both glycosylase and AP lyase activity).

Including a thermophilic glycosylase in combination with the thermophilic phosphatase provides even further improved sequencing metrics, in particular reduced error rates during the second sequencing read, as well as further reductions in overall run time.

The resynthesis kit may comprise the thermophilic glycosylase at a concentration of about 0.01 μM to about 1000 μM, about 0.02 μM to about 100 μM, about 0.05 μM to about 50 μM, about 0.1 μM to about 20 μM, or about 0.2 μM to about 10 μM.

The thermophilic glycosylase may preferably be derived from a thermophile, wherein the thermophile is of the genus Methanocaldococcus (Methanococcus). In one embodiment, the thermophile may be Methanocaldococcus jannaschii.

In a preferred embodiment, the thermophilic glycosylase may comprise the following sequence, or a functional variant or functional fragment thereof:

(SEQ ID NO: 2)
MMLIKKIEELKNSEIKDIIDKRIQEFKSFKNKSNEEWFKELCFCILTAN
FTAEGGIRIQKEIGDGFLTLPREELEEKLKNLGHRFYRKRAEYIVLARR
FKNIKDIVESFENEKVAREFLVRNIKGIGYKEASHFLRNVGYDDVAIID
RHILRELYENNYIDEIPKTLSRRKYLEIENILRDIGEEVNLKLSELDLY
IWYLRTGKVLK

The thermophilic glycosylase may preferably have a denaturation temperature of above 40° C., above 45° C., above 50° C., above 55° C., above 60° C., above 65° C., above 70° C., above 75° C., above 80° C., above 85° C., above 90° C., or above 95° C. More preferably, the thermophilic phosphatase may have a denaturation temperature of between about 40° C. to about 200° C., about 45° C. to about 190° C., about 45° C. to about 180° C., about 55° C. to about 170° C., about 60° C. to about 160° C., about 65° C. to about 150° C., about 70° C. to about 140° C., about 75° C. to about 130° C., about 80° C. to about 120° C., about 85° C. to about 110° C., about 90° C. to about 105° C., or about 95° C. to about 100° C.

In a further embodiment, the resynthesis kit includes an exonuclease. In some embodiments, the exonuclease includes a thermostable exonuclease. In some embodiments, the exonuclease includes a thermostable exonuclease derived from Pyrococcus abyssi. In some embodiments, the thermostable exonuclease derived from Pyrococcus abyssi comprises the following sequence, or a functional variant or functional fragment thereof:

(SEQ ID NO: 12)
FLVKFSDPYNYDLSGRQAILDEINLAIELAKRTKPDVVHLDSTLGGIEV
RKLDEPTIEALSISDKGKEVWKDLSKDVQPLAKKFWEETGIEILAIGKS
SVPVRIAEIYSGIFSVKWAIEEVKKGHKDHLIIGLPRYMGVEIRDGKIT
GRTLDPREGNLYGEISIEVPNNIKWEVYPNPLVRRFHIFEIWGEY

In some embodiments, the exonuclease includes a thermostable exonuclease derived from Pyrococcus furiosus. In some embodiments, the thermostable exonuclease derived from Pyrococcus furiosus comprises the following sequence, or a functional variant or functional fragment thereof:

(SEQ ID NO: 13)
MGSSHHHHHHSSGLVPRGSHMRIVAADTGGAVLDETFEPIGLIATVAVL
VEKPYRSAKEVMVKYANPYDYDLTGRQAIRDEVLLAIELARKVKPDVIH
LDSTLGGIELRKLDEPTIDALGISDKGKEVWKELSKDLQPLARKFWEET
NIEIVAIGKSSVPVRIAEIYAGIYSAKWGIENVEKEGHLIIGLPRYMEV
NIKDGKIIGRSLDPREGGLYGSAEVSVPEGVKWEIYPNPVARRFMIFEI
FSKR

The thermostable exonuclease can preferably be in a concentration range of between about 1 ug/ml and about 100 ug/ml, in the resynthesis kit, for example, between about 1 ug/ml and 10 ug/ml, between about 10 ug/ml and 20 ug/ml, between about 20 ug/ml and 30 ug/ml, between about 30 ug/ml and 40 ug/ml, between about 40 ug/ml and 50 ug/ml, between about 50 ug/ml and 60 ug/ml, between about 60 ug/ml and 70 ug/ml, between about 70 ug/ml and 80 ug/ml, between about 80 ug/ml and 90 ug/ml, or between about 90 ug/ml and 100 ug/ml.

The thermostable exonuclease may preferably include a melting temperature above 100° C., above 101° C., above 102° C., above 103° C., above 104° C., above 105° C., above 106° C., above 107° C., above 108° C., above 109° C., above 110° C., above 111° C., above 112° C., above 113° C., above 114° C., above 115° C., above 116° C., above 117° C., above 118° C., above 119° C., or above 120° C. More preferably, the thermostable exonuclease may have a melting temperature of between about 100° C. to about 120° C., about 101° C. to about 119° C., about 102° C. to about 118° C., about 103° C. to about 117° C., about 104° C. to about 116° C., about 105° C. to about 115° C., about 106° C. to about 114° C., about 107° C. to about 113° C., or about 108° C. to about 112° C. In some embodiments, the thermostable exonuclease includes a melting temperature of about 108.3° C.

In some embodiments, the exonuclease functions to reduce background noise in sequencing reactions by cleaving excess primers post clustering and prior to read 1 and/or post resynthesis during the paired-end turn step in preparation for read 2.

As used herein, the term “functional variant” refers to a variant polypeptide sequence or part of the polypeptide sequence which retains the biological function of the full non-variant sequence. For example, a functional variant of a phosphatase is able to catalyse the conversion a 3′-phosphorylated (poly)nucleotide chain to provide a dephosphorylated version of the (poly)nucleotide chain, as defined herein for the term “phosphatase”; a functional variant of a glycosylase is able to catalyse the removal of a nitrogenous base from one of the nucleotides in a (poly)nucleotide chain by breaking a N-glycosidic bond, resulting in the formation of an apurinic/apyrimidinic site (AP site), and may further have AP lyase activity, as defined herein for the term “glycosylase”.

A functional variant also comprises a variant of the polypeptide of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a polypeptide sequence that does not affect the functional properties of the polypeptide are well known in the art. For example, the amino acid alanine, a hydrophobic amino acid, may be substituted by another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

As used in any aspect described herein, a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant amino acid sequence and preferably retains the catalytic activity of a phosphatase or a glycosylase as described above. The sequence identity of a variant can be determined using any number of sequence alignment programs known in the art. As an example, Emboss Stretcher from the EMBL-EBI may be used: https://www.ebi.ac.uk/Tools/psa/emboss_stretcher/ (using default parameters: pair output format, Matrix=BLOSUM62, Gap open=1, Gap extend=1 for proteins; pair output format, Matrix=DNAfull, Gap open=16, Gap extend=4 for nucleotides).

As used herein, the term “functional fragment” refers to a functionally active series of consecutive amino acids from a longer polypeptide or protein. For example, a functional fragment may retain the catalytic activity of a phosphatase or a glycosylase, as described herein.

The resynthesis kit may further comprise a recombinase. Preferably, the recombinase is a thermophilic recombinase. The recombinase may be provided separately from the thermophilic phosphatase and/or the polymerase. For example, the recombinase may be in a different container to the thermophilic phosphatase and/or the polymerase.

As used herein, the term “recombinase” may refer to an enzyme which can facilitate invasion of a target nucleic acid by a polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolysable analogs thereof) in a recombinase-facilitated amplification reagent to facilitate amplification. A mixture of recombinase and single-stranded binding (SSB) protein is particularly useful as SSB can further facilitate amplification. Recombinases may include, for example, RecA protein, the T4 uvsX protein, any homologous protein or protein complex from any phyla, or functional variants thereof. Eukaryotic RecA homologues are generally named Rad51 after the first member of this group to be identified. Other non-homologous recombinases may be utilised in place of RecA, for example, RecT or RecO.

The resynthesis kit may further comprise a single-stranded nucleotide binding protein. Preferably, the recombinase is a thermophilic single-stranded nucleotide binding protein. The single-stranded nucleotide binding protein may be provided separately from the thermophilic phosphatase and/or the polymerase. For example, the single-stranded nucleotide binding protein may be in a different container to the thermophilic phosphatase and/or the polymerase.

As used herein, the term “single-stranded nucleotide binding protein” may refer to any protein having a function of binding to a single stranded nucleic acid, for example, to prevent premature annealing, to protect the single-stranded nucleic acid from nuclease digestion, to remove secondary structure from the nucleic acid, or to facilitate replication of the nucleic acid. The term is intended to include, but is not necessarily limited to, proteins that are formally identified as Single Stranded Binding proteins by the Nomenclature Committee of the International Union of Biochemistry and Molecular Biology (NC-IUBMB). Exemplary single stranded binding proteins include, but are not limited to E. coli SSB, T4 gp32, T7 gene 2.5 SSB, phage phi 29 SSB, any homologous protein or protein complex from any phyla, or functional variants thereof.

The resynthesis kit may further comprise a nucleotide triphosphate (NTP). Preferably, the nucleotide triphosphate may be a deoxynucleotide triphosphate (dNTP). More preferably, the kit comprises a plurality of NTPs or dNTPs, and preferably a mixture—for example comprising a plurality of dATP, dGTP, dCTP and dTTP for DNA clustering/synthesis or ATP, GTP, CTP and UTP for RNA clustering/synthesis. In one embodiment, the concentration of dNTPs may be between 0.1 and 2 mM, preferably between 0.2 to 1.5 mM, more preferably between 0.3 to 1.2 mM, even more preferably between 0.3 to 0.6 mM; for example, the concentration may be selected from 0.3 mM, 0.6 mM and 1.2 mM. The nucleotide triphosphate may be provided separately from the thermophilic phosphatase and/or the polymerase. For example, the nucleotide triphosphate may be in a different container to the thermophilic phosphatase and/or the polymerase.

As used herein, the term “nucleotide triphosphate” may refer to a molecule containing a nitrogenous base (e.g. adenine, thymine, cytosine, guanine, uracil) bound to a 5-carbon sugar (e.g. ribose or deoxyribose), with three phosphate groups bound to the sugar.

As used herein, the term “deoxynucleotide triphosphate” or (dNTPs) may refer to a molecule containing a nitrogenous base (e.g. adenine, thymine, cytosine, guanine, uracil) bound to deoxyribose, with three phosphate groups bound to the deoxyribose.

The resynthesis kit may further comprise an ATP-generating substrate. The ATP-generating substrate may be provided separately from the thermophilic phosphatase and/or the polymerase. For example, the ATP-generating substrate may be in a different container to the thermophilic phosphatase and/or the polymerase.

As used herein, the term “ATP-generating substrate” may refer to any substrate that is able to react with ADP to form ATP. Examples of ATP-generating substrates include creatine phosphate (CP).

The resynthesis kit may further comprise an ATP-generating enzyme. Preferably, the ATP-generating enzyme is a thermophilic ATP-generating enzyme. The ATP-generating enzyme may be provided separately from the thermophilic phosphatase and/or the polymerase. For example, the ATP-generating enzyme may be in a different container to the thermophilic phosphatase and/or the polymerase.

As used herein, the term “ATP-generating enzyme” may refer to any enzyme that is able to catalyse a reaction of ADP to form ATP. Examples of ATP-generating enzymes include creatine kinase.

The ATP-generating substrate as described herein may be paired with an appropriate ATP-generating enzyme that catalyses the reaction of that ATP-generating substrate with ADP to form ATP. Thus, in some preferred embodiments, the resynthesis kit may comprise creatine phosphate (CP) and creatine kinase.

The resynthesis kit may comprise at least one selected from the group consisting of: a recombinase, a single-stranded nucleotide binding protein, a polymerase, nucleotide triphosphates (NTPs), an ATP-generating substrate and an ATP-generating enzyme. The resynthesis kit may comprise at least one selected from the group consisting of: a recombinase, a single-stranded nucleotide binding protein, nucleotide triphosphates (NTPs), an ATP-generating substrate and an ATP-generating enzyme. Preferably, the kit may comprise at least two selected from the group consisting of: a recombinase, a single-stranded nucleotide binding protein, nucleotide triphosphates (NTPs), an ATP-generating substrate and an ATP-generating enzyme. More preferably, the kit may comprise at least three selected from the group consisting of: a recombinase, a single-stranded nucleotide binding protein, nucleotide triphosphates (NTPs), an ATP-generating substrate and an ATP-generating enzyme. Even more preferably, the kit may comprise at least four selected from the group consisting of: a recombinase, a single-stranded nucleotide binding protein, nucleotide triphosphates (NTPs), an ATP-generating substrate and an ATP-generating enzyme. One or more (e.g. each of these components) may be provided separately from the thermophilic phosphatase and/or the polymerase. For example, one or more (e.g. each of these components) may be in a different container to the thermophilic phosphatase and/or the polymerase.

Preferably, the resynthesis kit further comprises at least one selected from the group comprising a recombinase, NTPs and a single stranded nucleotide binding (SSB) protein. More preferably, the kit further comprises at least two selected from the group comprising a recombinase, NTPs and a single stranded nucleotide binding (SSB) protein. One or more (e.g. each of these components) may be provided separately from the thermophilic phosphatase and/or the polymerase. For example, one or more (e.g. each of these components) may be in a different container to the thermophilic phosphatase and/or the polymerase.

Preferably, the resynthesis kit may comprise a recombinase, NTPs and a single stranded nucleotide binding (SSB) protein. One or more (e.g. each of these components) may be provided separately from the thermophilic phosphatase and/or the polymerase. For example, one or more (e.g. each of these components) may be in a different container to the thermophilic phosphatase and/or the polymerase.

Preferably, the resynthesis kit may comprise a recombinase, a single-stranded nucleotide binding protein, nucleotide triphosphates (NTPs), an ATP-generating substrate and an ATP-generating enzyme. One or more (e.g. each of these components) may be provided separately from the thermophilic phosphatase and/or the polymerase. For example, one or more (e.g. each of these components) may be in a different container to the thermophilic phosphatase and/or the polymerase.

In some embodiments, the resynthesis kit may also comprise a nucleic acid template. The nucleic acid template may also comprise the adaptor sequences described herein, where preferably the adaptor sequences comprise at least one of P5, P5′, P7 and P7′, the sequences of which are described below.

The resynthesis kit may further comprise excipients. The excipients may be included within a composition comprising at least one of the kit components described herein (e.g. the thermophilic phosphatase, the polymerase, the glycosylase, the recombinase, the single-stranded nucleotide binding protein, the nucleotide triphosphates (NTPs), the ATP-generating substrate and/or the ATP-generating enzyme). In other embodiments, the excipients may be provided separately from the other kit components (e.g. the thermophilic phosphatase, the polymerase, the glycosylase, the recombinase, the single-stranded nucleotide binding protein, the nucleotide triphosphates (NTPs), the ATP-generating substrate and/or the ATP-generating enzyme). For example, the excipient(s) may be in a different container to the other kit components (e.g. the thermophilic phosphatase, the polymerase, the glycosylase, the recombinase, the single-stranded nucleotide binding protein, the nucleotide triphosphates (NTPs), the ATP-generating substrate and/or the ATP-generating enzyme). Suitable excipients may include surfactants, such as anionic surfactants, including alkyl sulfates (e.g. ammonium lauryl sulfate, sodium lauryl sulfate, sodium laureth sulfate, sodium myreth sulfate, sodium docusate), alkyl sulfonates (e.g. perfluorooctanesulfonate, perfluorobutanesulfonate), alkyl phosphates (e.g. alkyl-aryl ether phosphates, alkyl ether phosphates) and alkyl carboxylates (e.g. sodium stearate, sodium lauroyl sarcosinate, perfluorononanoate, perfluorooctanoate); cationic surfactants, including quaternary ammonium salts (e.g. cetrimonium bromide, cetylpyridinium chloride, benzalkonium chloride, benzethonium chloride, dimethyldioctadecylammonium chloride, dioctadecyldimethylammonium bromide); non-ionic surfactants, including fatty alcohol ethoxylates, alkylphenol ethoxylates, fatty acid ethoxylates, ethoxylated amines or fatty acid amides, poloxamers, polysorbates, (e.g. polyethylene glycol sorbitan alkyl esters (Tween)). Further excipients may include enzyme stabilisers, such as dithiothreitol (DTT), tris(2-carboxyethyl)phosphine (TCEP) and 2-mercaptoethanol (BME). Still further excipients may include molecular crowding agents such as polyethylene glycol (PEG), dextrans and epichlorohydrin-sucrose polymers (e.g. Ficoll).

The resynthesis kit may further comprise one or more agents for use in preparing a template nucleic acid sequence for clustering and sequencing (i.e. library preparation agents). In one embodiment, the kit may further comprise adaptor sequences. The adaptor sequences may be configured such that they can be ligated onto a nucleic acid template to be sequenced. In some preferred embodiments, the kit may comprise a first adaptor sequence that comprises a sequence according to SEQ ID NO. 3 (P5) or a variant or fragment thereof In other preferred embodiments, the kit may comprise a second adaptor sequence that comprises a sequence according to SEQ ID NO. 4 (P7) or a variant or fragment thereof In other preferred embodiments, the kit may comprise a third adaptor sequence that comprises a sequence according to SEQ ID NO. 5 (P5′) or a variant or fragment thereof. In other preferred embodiments, the kit may comprise a fourth adaptor sequence that comprises a sequence according to SEQ ID NO. 6 (P7′) or a variant or fragment thereof. More preferably, the kit may comprise at least two of the group selected from the first adaptor sequence, the second adaptor sequence, the third adaptor sequence and the fourth adaptor sequence. Even more preferably, the kit may comprise at least three of the group selected from the first adaptor sequence, the second adaptor sequence, the third adaptor sequence and the fourth adaptor sequence. Yet even more preferably, the kit may comprise the first adaptor sequence, the second adaptor sequence, the third adaptor sequence and the fourth adaptor sequence. The adaptor sequence(s) (e.g. each of the adaptor sequence(s)) may be provided separately from the thermophilic phosphatase and/or the polymerase. For example, the adaptor sequence(s) (e.g. each of the adaptor sequence(s)) may be in a different container to the thermophilic phosphatase and/or the polymerase.

The resynthesis kit may further comprise a metal cofactor composition. The metal cofactor may be configured to activate one or more enzymes in the resynthesis kit. For example, the metal cofactor may be configured to activate the recombinase and/or the polymerase. Preferably, the metal cofactor composition comprises magnesium ions (e.g. magnesium acetate, magnesium chloride). The metal cofactor composition may be provided separately from the thermophilic phosphatase and/or the polymerase. For example, the metal cofactor composition may be in a different container to the thermophilic phosphatase and/or the polymerase.

The resynthesis kit may further comprise a solid support, preferably a flow cell. Preferably lawn primers (P5 and P7) are immobilised on the flow cell as described in detail above.

The resynthesis kit may further comprise instructions for use of the kit in resynthesis of a nucleic acid template, or pairwise sequencing of a nucleic acid template. For example, the instructions may take the form of a manual, a pamphlet or user guide.

In a further embodiment, the present disclosure is directed to a resynthesis kit comprising a thermophilic glycosylase and a polymerase.

Preferably, the thermophilic glycosylase is as described herein.

Preferably, the polymerase is as described herein.

In a further embodiment, the present disclosure is directed to use of a resynthesis kit as described herein, in resynthesis of a nucleic acid template, or pairwise sequencing of a nucleic acid sequence.

### Lyophilization of Compositions

As used herein, the term “lyophilization” refers to a process in which a composition is frozen followed by dehydration of the product at low pressure. Lyophilization results in transition of the composition from a solid phase directly to a gas phase, without passing through a liquid phase. As used herein, methods of lyophilization include shelf-freeze-drying and spray-freeze-drying to produce lyophilized cakes and/or lyophilized microspheres.

In an aspect, any phosphatase described herein is lyophilized. In some embodiments, a lyophilized formulation that includes a phosphatase includes a salt, an exonuclease, a detergent, and any one or more of magnesium chloride, acetate, or sulfate. In embodiments, the salt includes sodium chloride. In embodiments, the salt includes potassium chloride. In embodiments, the exonuclease includes Pyrococcus abyssi alkaline phosphatase (PAAP). In embodiments, the detergent includes polysorbate 20 or tricosaethylene glycol dodecyl ether.

In some embodiments, the lyophilized formulation that includes a phosphatase includes (i) a buffer, (ii) a salt, (iii) (2-Hydroxylpropyl)-β-Cyclodextrin (HPBCD), (iv) (tris(2-carboxyethyl(phosphine) (TCEP), (v) polysorbate 20 or tricosaethylene glycol dodecyl ether, (vi) trehalose, (vii) zinc chloride or zinc acetate, (viii) PAAP, (ix) magnesium chloride, acetate or sulfate. In some embodiments, the buffer includes a tris or bis tris propane buffer. In some embodiments, the buffer includes a pH between about 7.5 and 9. In some embodiments, the salt includes potassium chloride or sodium chloride. In some embodiments, the salt concentration is between 50 mM and 200 mM. In some embodiments, the concentration of the HPBCD is between 0.1% and 4% w/v. In some embodiments, the concentration of the TCEP is between 0.5 mM and 5 mM. In some embodiments, the concentration of the polysorbate 20 or tricosaethylene glycol dodecyl ether is between 0.005% and 0.1% w/v. In some embodiments, the concentration of the trehalose is between 4% and 20% w/v. In some embodiments, the concentration of the zinc chloride or zinc acetate is between a 1 molar and 3 molar ratio to the PAAP concentration. In some embodiments, the concentration of PAAP is between 0.002 mg/ml and 1 mg/ml. In some embodiments, the concentration of the chloride, acetate, or sulfate is between 2 mM and 20 mM.

In an aspect, any glycosylase described herein is lyophilized. In some embodiments, the lyophilized formulation that includes a glycosylase includes (i) a salt, (ii) oxoguanine glycosylase (OGG), and (iii) trehalose or trehalose and raffinose. In some embodiments, the salt includes either sodium hydroxide or potassium hydroxide.

In some embodiments, the lyophilized formulation that includes a glycosylase includes (i) a buffer, (ii) a salt, (iii) HPBCD, (iv) TCEP, (v) polysorbate 20 or tricosaethylene glycol dodecyl ether, (vi) trehalose, (vii) raffinose, (viii) OGG, and (ix) polyvinylpyrriolidone (PVP). In some embodiments, the buffer includes tris buffer at a pH between 7.5 and 9. In some embodiments, the salt includes potassium chloride or sodium chloride. In some embodiments, the salt concentration is between 20 mM and 300 mM. In some embodiments, the HPBCD is between 0.1% and 3.0% w/v. In some embodiments, the polysorbate 20 or trisosaethylene glycol dodecyl ether is between 0.005% and 0.1%. In some embodiments, the trehalose is between 4% and 20% w/v. In some embodiments, the raffinose is between 0.1% and 3% w/v. In some embodiments, the OGG is between 0.1 uM and 500 uM. In some embodiments, the PVP is between 0.1% and 2%.

In an aspect, any thermostable exonuclease described herein is lyophilized. In some embodiments, a lyophilized formulation that includes a thermostable exonuclease includes (i) a buffer, (ii) a salt, (iii) trehalose, (iv) hydroxypropyl-beta-cyclodextrin, (v) magnesium acetate, sulfate, or chloride, (vi) tricosaethylene glycol dodecyl either or polysorbate 20, and (vii) the thermostable exonuclease. In some embodiments, the buffer includes BisTris Propane or Tris. In some embodiments, the pH of the buffer is between about 7.5 and 9. In some embodiments, the salt is sodium hydroxide or potassium hydroxide. In some embodiments, the concentration of the salt is between 50 mM and 200 mM. In some embodiments, the concentration of trehalose is between 4% and 30% w/v. In some embodiments, the concentration of the magnesium acetate, sulfate, or chloride is between 2 mM and 50 mM. In some embodiments, the concentration of the tricosaethylene glycol dodecyl either or polysorbate 20 is between 0.005% and 0.2%.

### Methods

In a further embodiment, the present disclosure is directed to a method of conducting resynthesis of a nucleic acid sequence, wherein the method comprises:
removing a blocking phosphate group from a blocked primer using thermophilic phosphatase to form a deblocked primer; and
forming a second nucleic acid template extending from the deblocked primer using a polymerase and a first nucleic acid template.

As used herein, the term “blocking phosphate group” may refer to a phosphate residue that is present at an end of a (poly)nucleotide chain (for example, at a 3′ end of a (poly)nucleotide chain). The blocking phosphate group may prevent chain extension from that end of the (poly)nucleotide chain under reaction conditions used during amplification and/or sequencing (e.g. reactions utilising a polymerase).

As used herein, the term “blocked primer” may refer to a primer (e.g. a P5 or P7 primer as described herein) in an inactive form which is unable to undergo chain extension from a blocked end of its (poly)nucleotide chain (for example, at a 3′ end of the (poly)nucleotide chain) under reaction conditions used during amplification and/or sequencing (e.g. reactions utilising a polymerase). A blocked primer may comprise a blocking group for this purpose, for example a blocking phosphate group. A blocked primer may be changed into its active form by removal of the blocking group, thus forming a “deblocked primer”, which is able to undergo chain extension from the (previously blocked) end of its (poly)nucleotide chain.

As used herein, the term “first nucleic acid template” may refer to a (poly)nucleotide chain that has been previously sequenced in a first sequencing read. A first end of the first nucleic acid template may be covalently attached to an adaptor sequence (e.g. P5′ or P7′) which is complementary to the blocked primer (e.g. a P5 or P7 primer as described herein), and is therefore able to bind to the blocked primer by base-pairing. As the first nucleic acid template has been previously sequenced, a second end of the first nucleic acid template may be covalently attached to another adaptor sequence (e.g. P7′ or P5′, different from the adaptor sequence attached to the first end of the first nucleic acid template).

Where solid supports are used, the second end of the first nucleic acid template may be covalently attached to a first immobilised primer (e.g. P7 or P5 primer as described herein, wherein the first immobilised primer is different from the blocked primer). In such a case, the first nucleic acid template constitutes a first immobilised nucleic acid template.

As used herein, the term “second nucleic acid template” may refer to a (poly)nucleotide chain that is to be sequenced in a second sequencing read. The second nucleic acid template may be complementary to the first nucleic acid template. One end of the second nucleic acid template may be covalently attached to the blocking primer.

Where solid supports are used, the blocking primer may constitute a second immobilised primer (e.g. P5 or P7 primer, different from the first immobilised primer). In such a case, the second nucleic acid template constitutes a second immobilised nucleic acid template.

The thermophilic phosphatase may be a thermophilic phosphatase as defined herein.

The polymerase may be a polymerase as defined herein.

Preferably, the blocked primer and/or deblocked primer may be immobilised on a solid support, preferably wherein the solid support is a flow cell.

Preferably, the step of forming the second nucleic acid template extending from the deblocked primer is conducted using bridge amplification.

In a preferred embodiment, the method further comprises a step of detaching the first nucleic acid template by using a thermophilic glycosylase.

The thermophilic glycosylase may be a thermophilic glycosylase as defined herein.

The thermophilic glycosylase allows a covalent bond to be broken in the first nucleic acid template, thereby cutting the (poly)nucleotide chain. The covalent bond may be located the first or second end of the first nucleic acid template, preferably the second end. This allows the first nucleic acid template to become detached (in particular in cases where a solid support is used), and thereby allow it to become dehybridised from the second nucleic acid template. The first nucleic acid template may then be washed away.

Preferably, the method is conducted isothermally. In other words, each of the steps in the method of conducting resynthesis of a nucleic acid sequence may be conducted at the same temperature.

In preferred embodiments, the method is conducted at a temperature of about 50° C. to about 75° C., preferably about 55° C. to about 70° C., or more preferably about 60° C. to about 65° C. For example, the method may be conducted at a temperature of about about 50° C., about 55° C., about 60° C., about 65° C., about 70° C., or about 75° C., preferably about 65° C.

In a further embodiment, the present disclosure is directed to a method of sequencing a nucleic acid sequence by pairwise sequencing, wherein the method comprises:
sequencing a first nucleic acid template;
conducting resynthesis of a nucleic acid sequence using a method as described herein to form a second nucleic acid template; and
sequencing the second nucleic acid template.

Preferably, the step of sequencing the first nucleic acid template and/or the step of sequencing the second nucleic acid template may be conducted using a sequencing-by-synthesis technique or a sequencing-by-ligation technique.

Preferably, the method comprises using a resynthesis kit as described herein.

The present disclosure will now be described by way of the following non-limiting examples.

### EXAMPLES

### Materials and Methods:

Preparation of Pyrococcus abyssi Alkaline Phosphatase (PAAP):

The PAAP gene was cloned into a pET15b vector, which harbors an ampicillin resistance gene (50 μg/ml). The plasmid was transformed into BL21 cells supplemented with kanamycin at 50 μg/ml and grown to an optical density of 0.6. The cells were induced with 0.5 mM IPTG and induction proceeded at 18° C. for 18 hrs. Cells were harvested and lysed with 3000 U/ml of ReadyLyse and 100 U/ml of Omincleave. Clarified lysate was heat treated (Ht Tx) for 80° C. for 70 min. Upon cooling for 60 min on ice, the lysate was centrifuged and the lysate was applied to a HisTrap column with a gradient elution fractions were collected and pooled. HisTrap pooled elution samples were applied to an anion exchange column and with a gradient elution fractions were pooled and dialyzed against a 200 mM NaCl, 50 mM Tris pH 7.5, and 50% glycerol buffer.

### Properties of PAAP Protein:

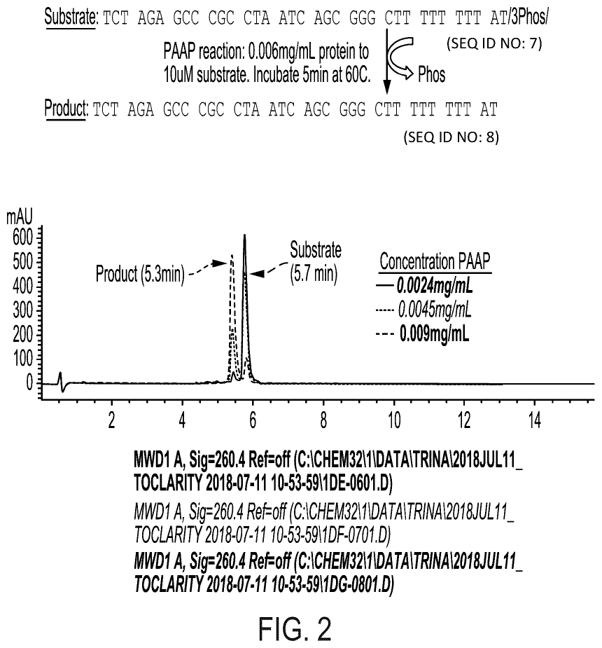

The PAAP protein is active on single-stranded nucleic acid substrates with a 3′ phosphate modified oligo. 50 μl reaction mixtures contain 10 μM of substrate with 0.006 mg/mL (˜0.1 μM) PAAP, which were incubated at 60° C. for 5 minutes. The reaction was terminated with the addition of 20 mM EDTA final concentration. The samples were resolved with reverse phase chromatography on a Clarity 1.7 μm oligo-MS 100 A, LC column with a HAA gradient. The control sample, which has no PAAP protein present produced a single peak with a retention time of 5.7 minutes. Upon the addition of PAAP protein, a product peak with the 3′ phosphate cleaved appears at 5.3 minutes. The results of this in vitro activity testing are shown in FIG. 2.

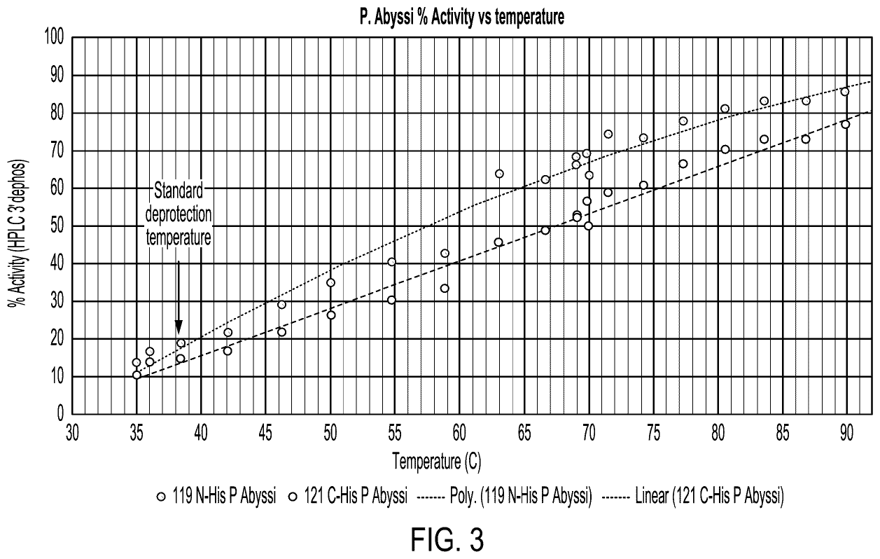

A concentration of 0.006 mg/mL (˜0.1 μM) was used in the in vitro activity assay above to yield ˜50% substrate and ˜50% dephosphorylated oligo (50% activity). Using this concentration, the enzyme was incubated using a temperature range from 35° C. to 95° C. using the standard 5 minute reaction. Samples were then immediately quenched with EDTA and analyzed on the HPLC clarity column. The results of testing of activity vs. temperature are shown in FIG. 3.

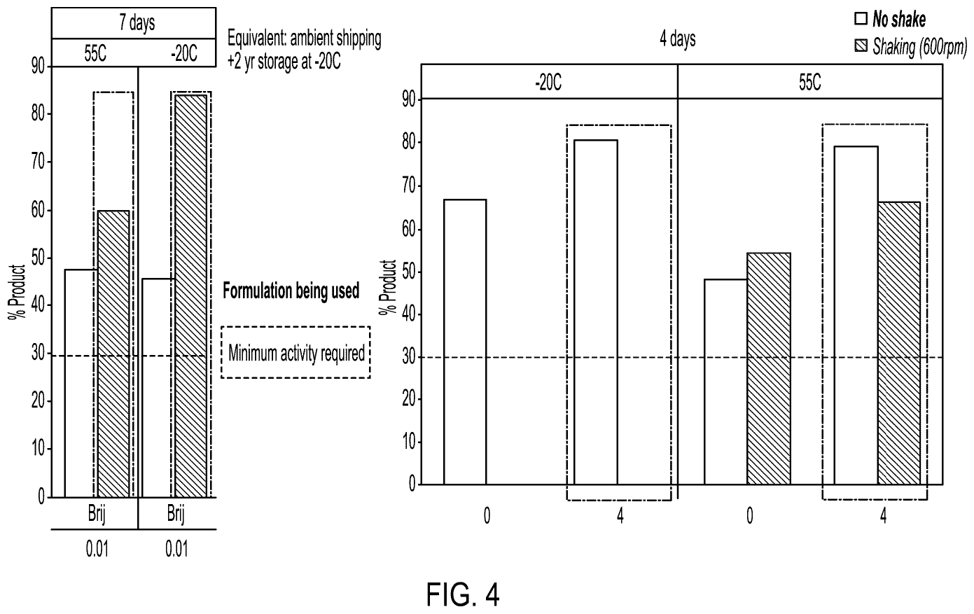

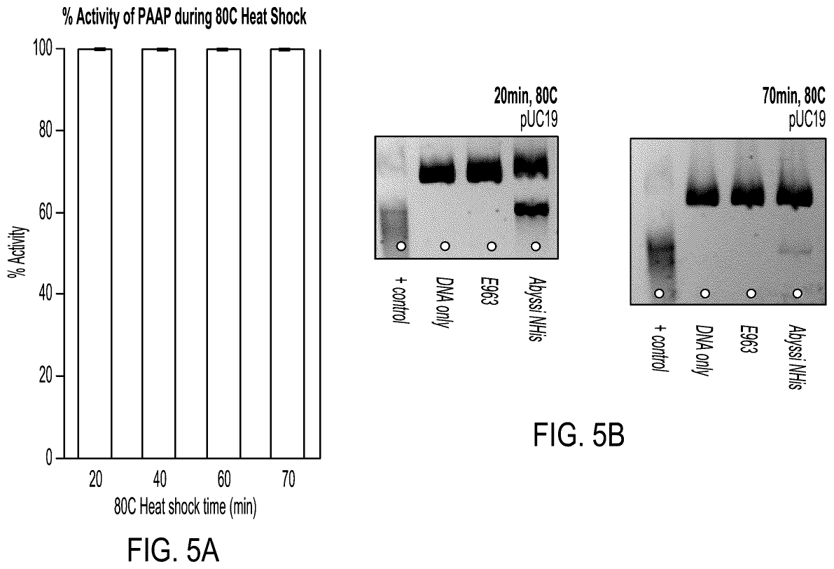

The PAAP protein was also found to be suitable in ambient shipping conditions (FIG. 4). Furthermore, PAAP protein was found to be stable towards heat shock, allowing for nuclease inactivation processes to be used (FIGS. 5A-5B).

Preparation of Methanocaldococcus (Methanococcus) jannaschii Oxoguanine Glycosylase (MjaOGG):

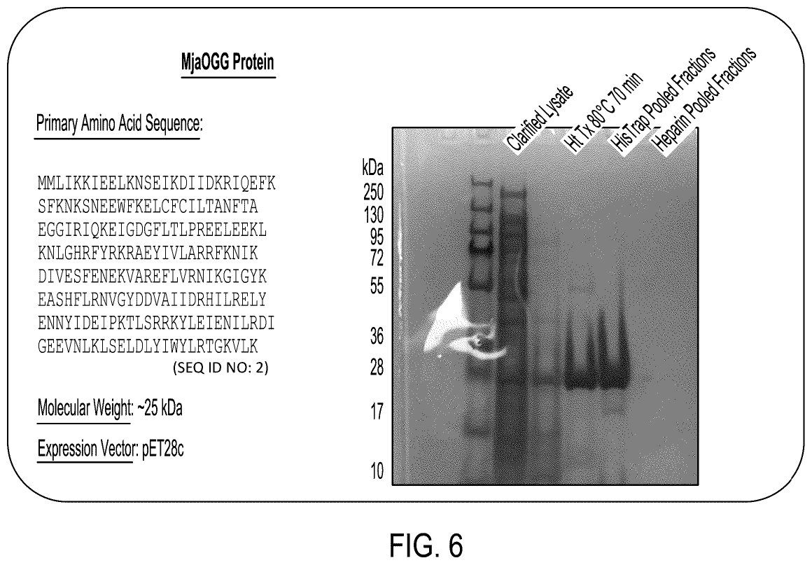

The MjaOGG gene was cloned into a pET28c vector, which harbours a kanamycin resistance gene (50 μg/ml). The plasmid was transformed into BL21 cells supplemented with kanamycin at 50 μg/ml and grown to an optical density of 0.8. The cells were induced with 0.1 mM IPTG and induction proceeded at 18° C. for 24 hrs. Cells were harvested and lysed with 3000 U/ml of ReadyLyse and 100 U/ml of Omincleave. Clarified lysate was heat treated (Ht Tx) for 80° C. for 70 min. Upon cooling for 60 min on ice, the lysate was applied to a HisTrap column with a gradient elution fractions were collected and pooled. HisTrap pooled elution samples were applied to a Heparin column and with a gradient elution fractions were pooled and dialyzed against a 300 mM NaCl, 50 mM Tris pH 7.5, 0.25 mM DTT and 50% glycerol buffer. Samples were run on a 4-12% SDSPAGE gel with a PageRuler Plus protein marker. The heparin pooled fractions were near homogeneous when stained with Coomassie Brilliant Blue dye (FIG. 6).

### Properties of MjaOGG Protein:

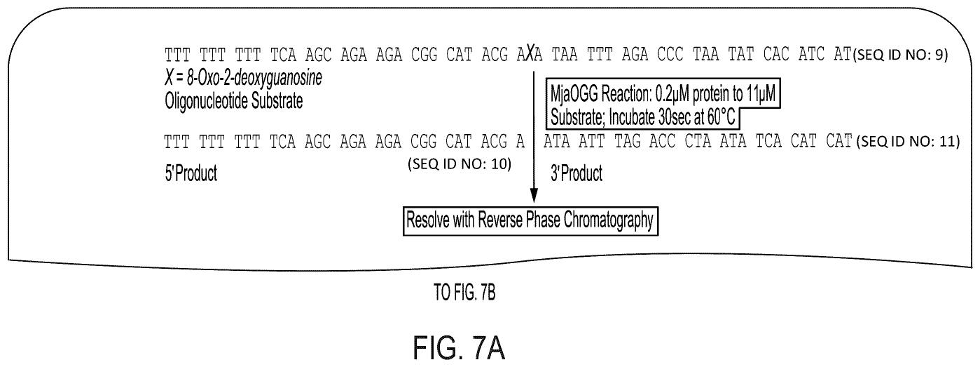

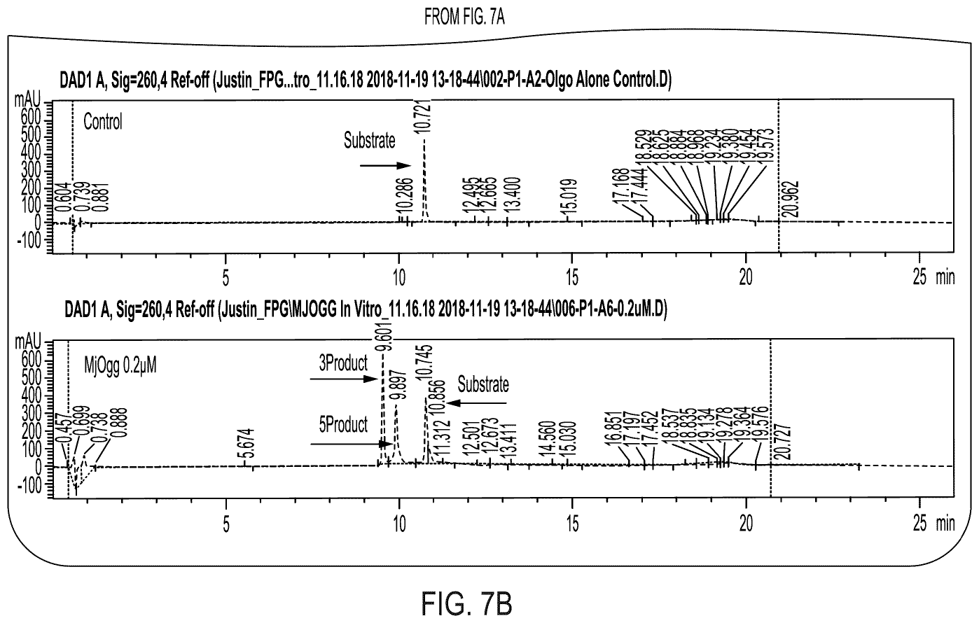

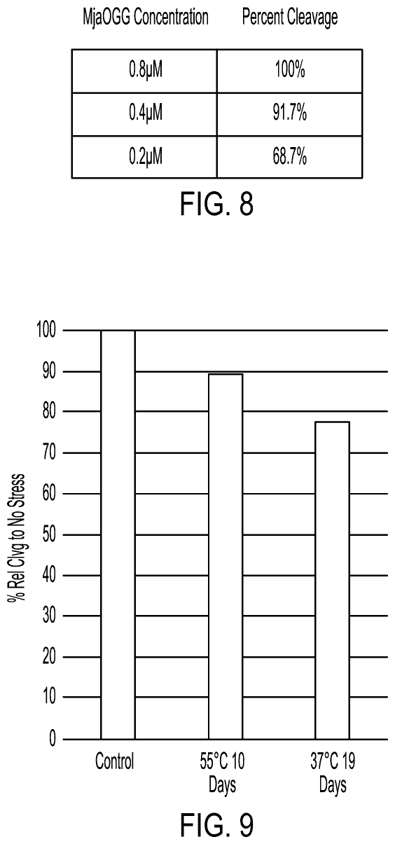

100 μl reaction mixtures contained 11 μM of substrate with 0.2 μM MjaOGG, which were incubated at 60° C. for 30 seconds. The reaction was terminated with the addition of 0.4 U/μl final concentration of proteinase K and incubated for 5 min. The samples were resolved with reverse phase chromatography on a Clarity 1.7 μm oligo-MS 100 A, LC column with a HAA gradient. The control sample, which had no MjaOGG protein present produced a single peak with a retention time of 10.72 min. Upon the addition of MjaOGG at 0.2 μM two product peaks are detected with the 3′ Product at 9.6 min and the 5′ Product at 9.89 min. The remaining substrate peak was detected at a retention time of 10.74 min. The results for in vitro activity are shown in FIGS. 7A-7B, whilst cleavage results for three test concentrations are shown in FIG. 8.

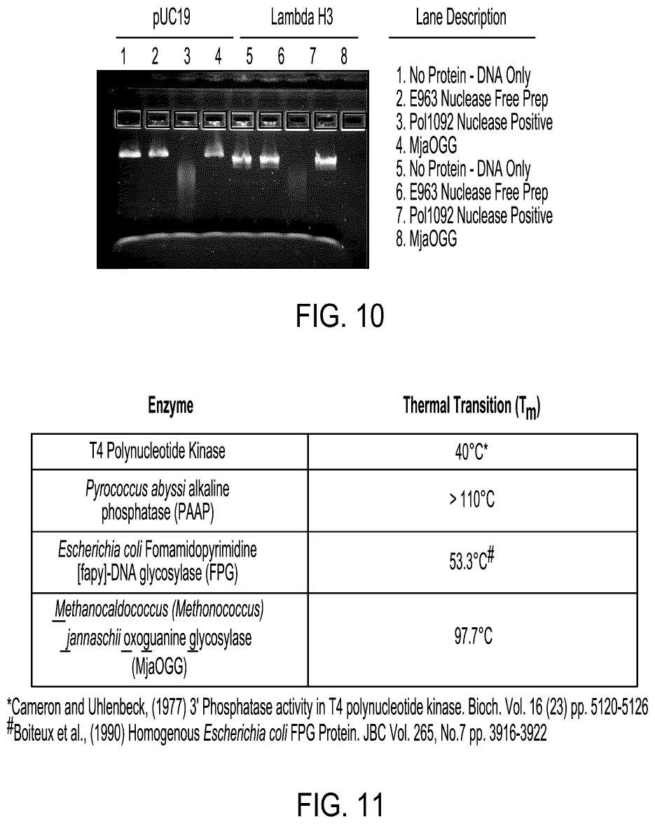

The MjaOGG protein was also found to be suitable in ambient shipping conditions (FIG. 9). Furthermore, MjaOGG protein was found to be stable towards heat shock, allowing for nuclease inactivation processes to be used (FIG. 10).

Comparative Testing for Mesophilic Enzymes vs. Thermophilic Enzymes:

Three different experiments were conducted to determine the effect of using thermophilic enzymes such as PAAP protein and MjaOGG protein on error rate during read 2.

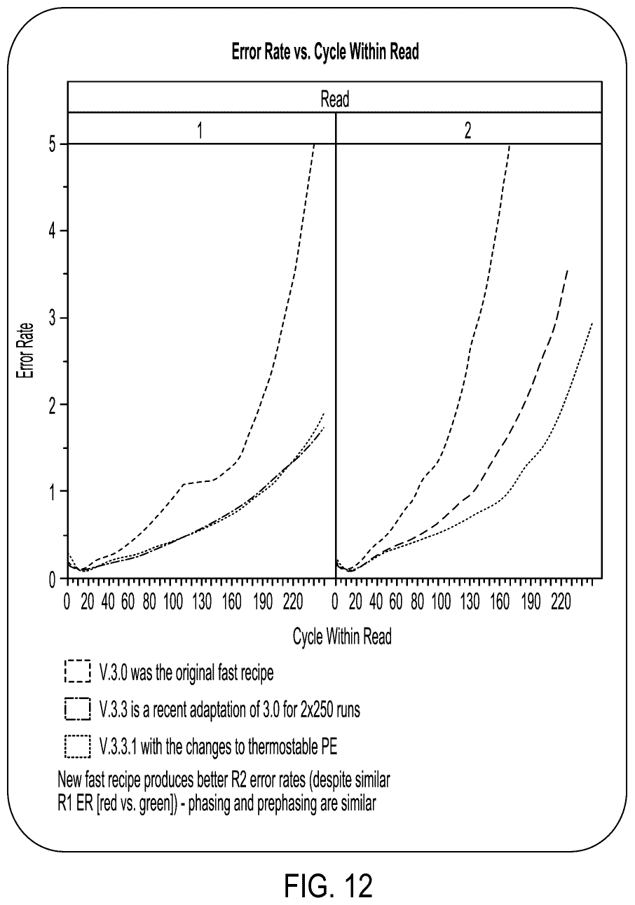

As comparative examples, an experiment was run using normal mesophilic enzyme recipes on the Illumina MiniSeq platform, whilst another experiment was run using a modified recipe used for 2×250 runs but still using mesophilic enzymes. These results are shown in FIG. 12 (blue—normal mesophilic enzyme recipe (V.3.0); green—modified recipe for 2×250 runs and mesophilic enzymes (V.3.3)).

A further experiment was run using the modified recipe for 2×250 runs but with PAAP protein (0.103 mg/mL; ˜2 μM) and MjaOGG protein (0.2 μM) (red). As shown in FIG. 12, read 1 error rates with the thermophilic enzymes were relatively similar to the corresponding mesophilic case. However, read 2 error rates with the thermophilic enzymes were reduced compared with the corresponding mesophilic case.

Accordingly, switching the currently used mesophilic enzymes to thermophilic enzymes such as PAAP protein and MjaOGG protein leads to improved primary metrics, such as reduced read 2 error rates. Furthermore, the total run time was reduced from 42 minutes and 14 seconds to 19 minutes and 44 seconds.

### Thermostable Exonucleases Used in Storage and in Sequencing Reactions:

Thermostable exonuclease from Pyrococcus abyssi and Pyroccus furiosus can be used to cleave excess flowcell primers post clustering and prior to read 1 and/or post resynthesis during the paired-end turn step in preparation for read 2, to reduce background noise/signal.

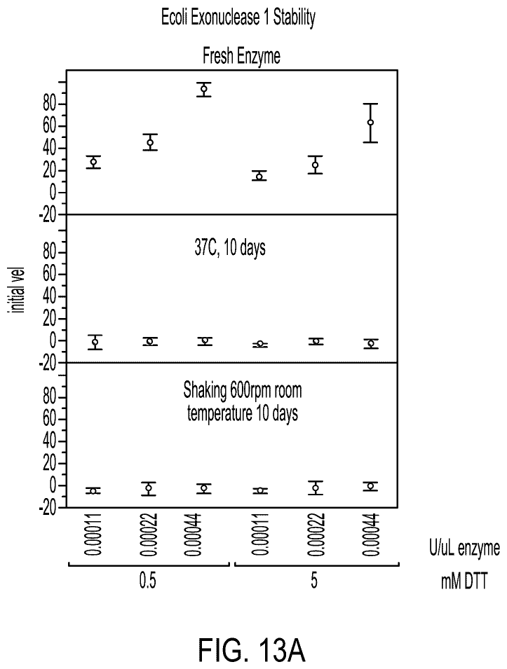

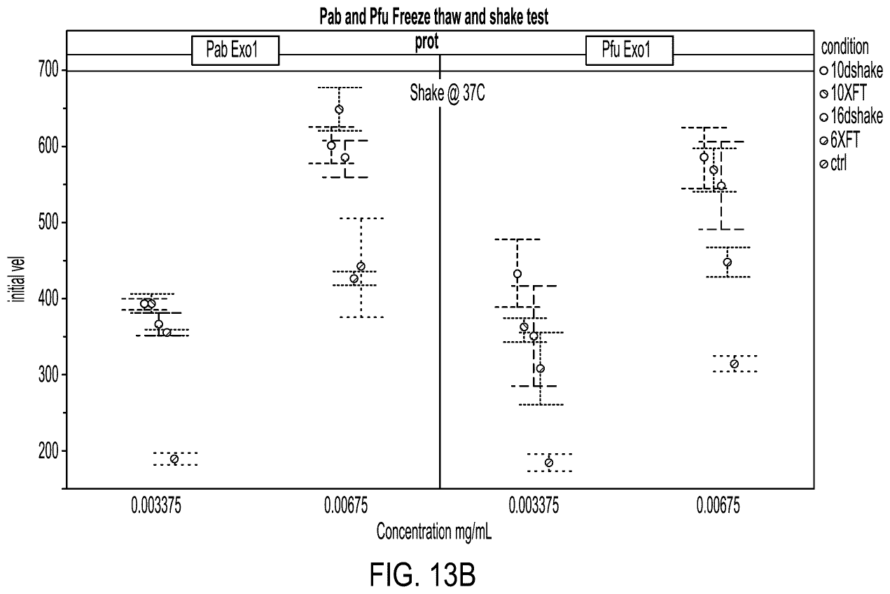

Storage of Exonucleases. Exonuclease derived from E. coli is insufficiently stable for ambient shipping conditions. As shown in the top panel of FIG. 13A, the exonuclease has high activity when it is fresh. However, as shown in the middle and lower panels of FIG. 13A, the activity of exonuclease derived from E. coli is significantly decreased after storage at 37° C. for 10 days and after shaking at 600 rpm at room temperature for 10 days. In contrast, as shown in FIG. 13B, thermostable exonucleases derived from Pyrococcus abyssi (SEQ ID NO: 12) and Pyroccus furiosus (SEQ ID NO: 13) retain their activity at 37° C. heating for 10 to 16 days with or without shaking. In addition, the Pyrococcus abyssi and Pyroccus furiosus thermostable exonucleases retain their activity through six freeze/thaw cycles. The thermostable exonucleases were each tested at two different concentrations: 0.003375 mg/ml and 0.00675 mg/ml.

Sequencing using Exonucleases. Activity of thermostable exonucleases in sequencing reactions were tested and compared to activity of exonuclease activity derived from E. coli, in sequencing reactions. The formulation that included thermostable exonuclease included the following elements:
Tris, pH 8.9
0.1% polysorbate 20
0.1 mM EDTA
0.05 mg/ml of Pyrococcus abyssi thermostable exonuclease (SEQ ID NO: 12)
45 mM of magnesium chloride
50 mM of potassium chloride
500 mM of trehalose

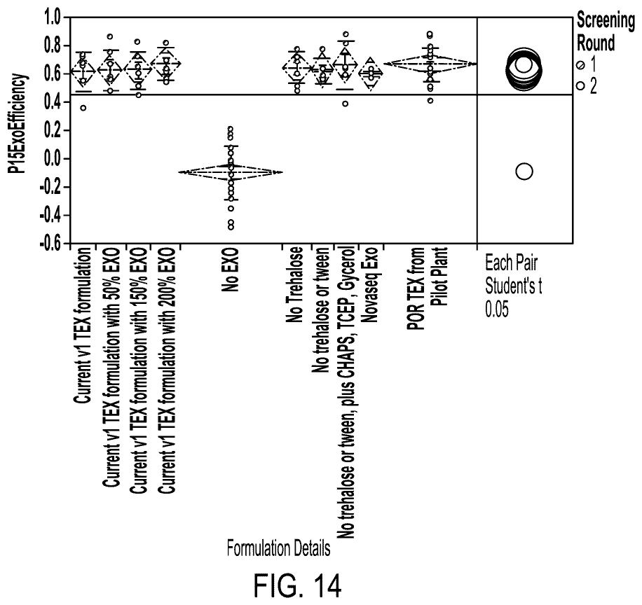

FIG. 14 shows that using thermostable exonuclease derived from Pyrococcus abyssi in sequencing reactions is as efficient as using exonuclease from E coli. TEX refers to thermostable exonuclease. Novaseq Exo refers to exonuclease derived from E. coli.

Lyophilization of Thermostable Exonuclease. Thermostable exonuclease activity was compared at various temperatures in both liquid and lyophilized formats. Thermostable exonuclease was lyophilized in the following formulation:
80 mM Bis Tris Propane pH 9
100 mM potassium chloride
392 mM trehalose
4 mM tris(2-carboxyethyl)phosphine
0.02% tricosaethylene glycol dodecyl ether
2% (2-Hydroxylpropyl)-β-Cyclodextrin
0.44 mg/ml of Pyrococcus abyssi thermostable exonuclease (SEQ ID NO: 12).

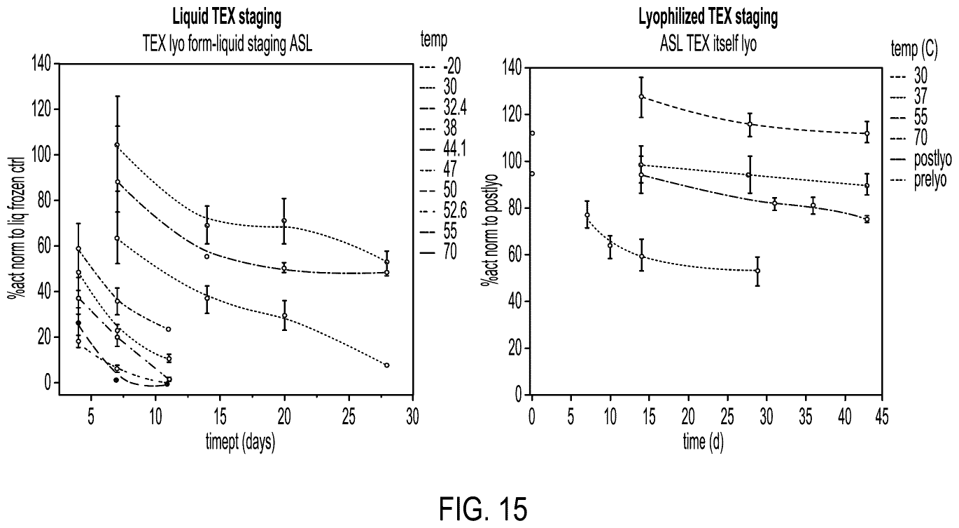

Prior to testing the lyophile was resuspended with 10 mM magnesium acetate. Samples were subjected to staging. Thermostable exonuclease activity was compared after staging in both liquid and lyophilized format, at different temperatures. FIG. 15 shows that the thermostable exonuclease has a significantly longer shelf life in the lyophilized format, which enables ambient storage.

Degradation Rates of Liquid Formulated Pyrococcus abyssi Alkaline Phosphatase (PAAP) and Lyophilized PAAP

PAAP was lyophilized in the following formulation:
80 mM BisTris Propane pH 9
100 mM potassium chloride
393 mM trehalose
4 mM tris(2-carboxyethyl)phosphine
0.02% tricosaethylene glycol dodecyl ether
2% (2-Hydroxylpropyl)-β-Cyclodextrin
8 uM Zinc Acetate
4 uM PAAP (SEQ ID NO: 1)

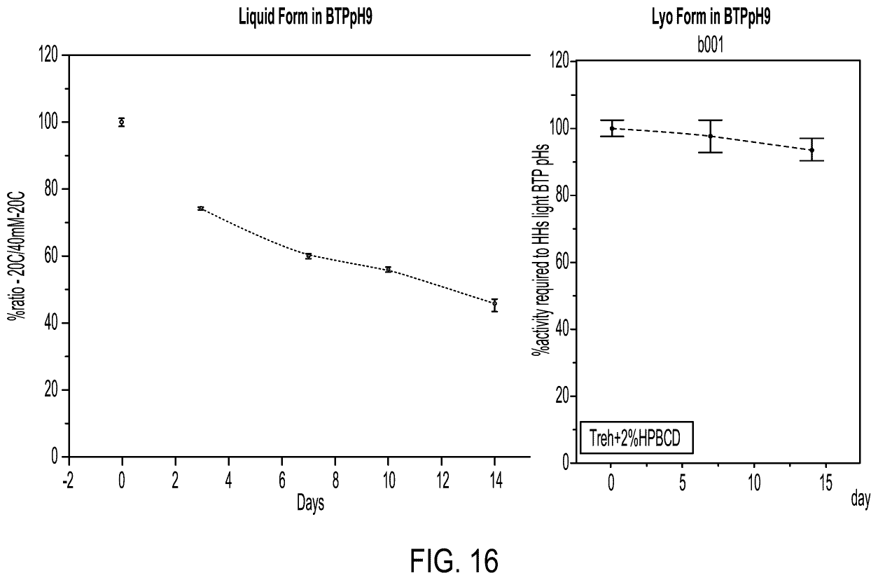

FIG. 16 shows that there was a lower degradation rate of PAAP when it is in the lyophilized format versus when PAAP is in the liquid format.

Screening Methanocaldococcus (Methanococcus) jannaschii oxoguanine glycosylase (MjaOGG). An activity screen was performed on MjaOGG (OGG) (SEQ ID NO: 2) in the presence of bulking lyophilized excipients, before and after heat stress. (2-Hydroxylpropyl)-β-Cyclodextrin (HPBCD), polyvinylpyrriolidone (PVP), raffinose, and trehalose were chosen as candidates for lyophilization. Each lyophilization formulation contained the following base components:
50 mM HEPES pH 7.5
300 mM sodium chloride
250 mM trehalose
0.05% polysorbate 20
7 uM OGG (SEQ ID NO: 2)

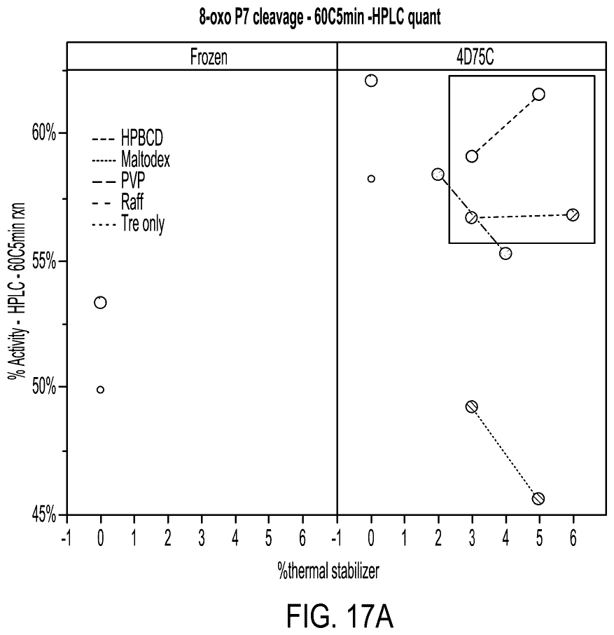

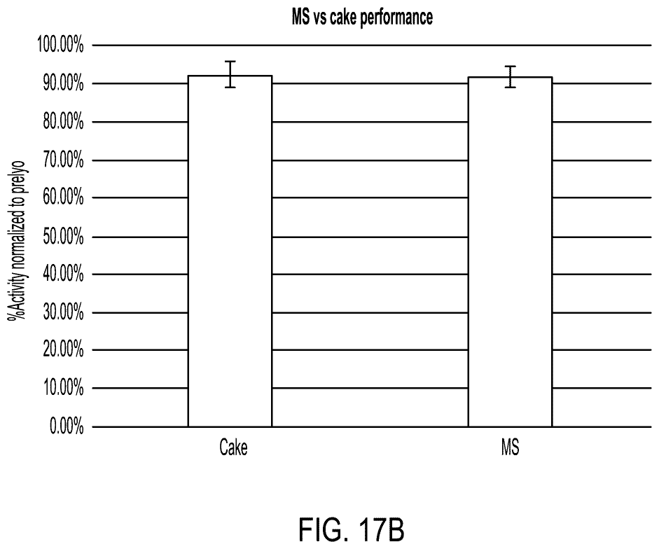

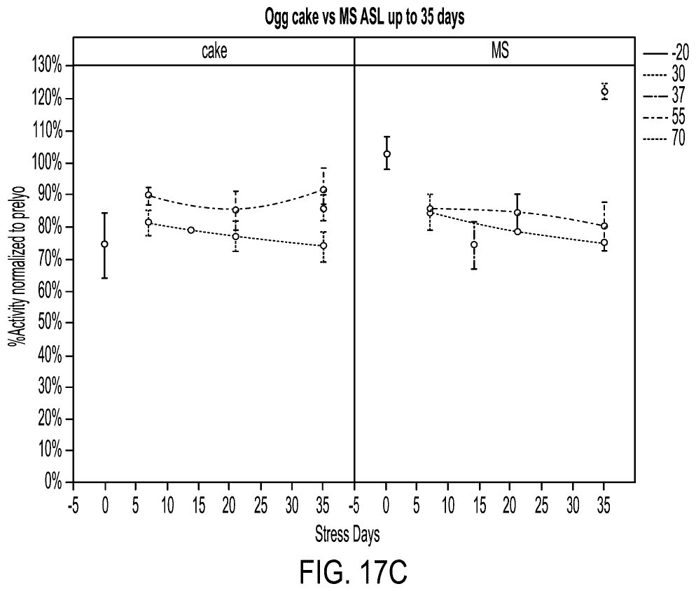

Five different formulations were tested. One formulation contained only the base components shown in the above formulation. Four additional formulations were tested: (i) each of the base components in the above formulation along with (2-Hydroxylpropyl)-β-Cyclodextrin (HPBCD); (ii) each of the base components in the above formulation along with Maltodex; (iii) each of the base components in the above formulation along with polyvinylpyrriolidone (PVP); and (iv) each of the base components in the above formulation along with raffinose. FIG. 17A shows that the activity of formulations (i), (iii), and (iv) resulted in high activity of the OGG enzyme.

Activity and stability of OGG were both analyzed in a microsphere format and a cake format. The lyophilized formulation tested included the following components:
50 mM Tris pH 7.5
300 mM sodium chloride
10.5% trehalose
3% 2-Hydroxylpropyl)-β-Cyclodextrin
3% raffinose
0.05% polysorbate 20

FIGS. 17B and 17C show that there was no difference in OGG activity between the cake format and microsphere format.

Lyophilized and liquid formats of PAAP (SEQ ID NO: 1) and OGG (SEQ ID NO: 2) were tested to see how the formats performed on primary and secondary sequencing metrics. The following formulations were tested:

### OLM2 (Liquid Formulation of OGG)

50 mM Tris pH 7.5
300 mM sodium chloride
0.05% polysorbate 20
0.5 nM OGG

### KLM2 (Lyophilized Formulation of OGG)

Tris pH 7.5
14.75% Trehalose
4% 2-Hydroxylpropyl)-β-Cyclodextrin
70 mM sodium chloride
0.05% tricosaethylene glycol dodecyl ether
1 uM OGG

### TRM (Liquid PAAP Formulation)

50 mM Tris pH 7.5
16 mM magnesium chloride
100 mM potassium chloride
0.02% trisosaethylene glycol dodecyl ether
1.5 mM tris(2-carboxyethyl)phosphine
0.4 mg/ml PAAP

### KRM (Lyophilized Formulation of PAAP)

50 mM Tris pH 8.8
50 mM potassium chloride
14.65% trehalose
4% (2-Hydroxylpropyl)-β-Cyclodextrin
2 mM tris(2-carboxyethyl)phosphine
0.02% tricosaethylene glycol dodecyl ether
8 uM zinc chloride
4 mM MgOAc

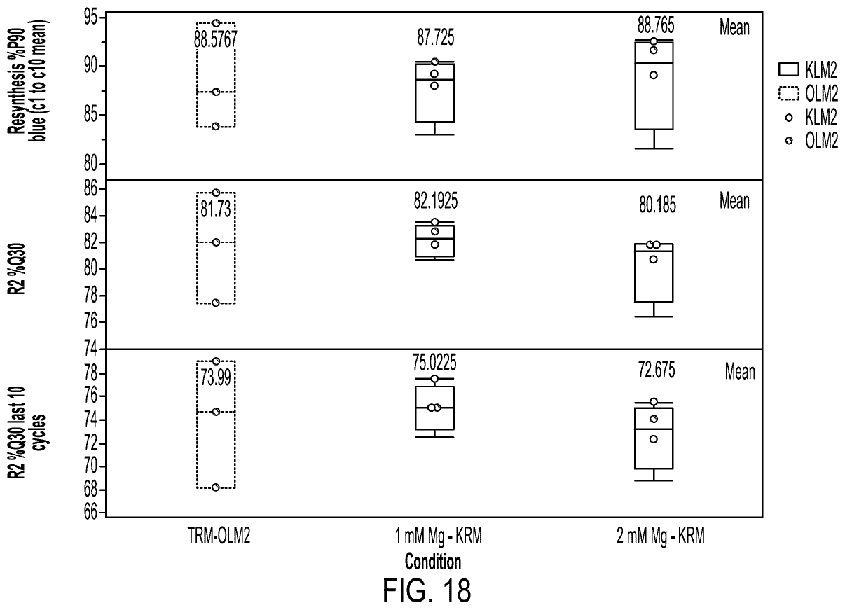

FIG. 18 shows that there was no difference in performance between the conditions in which 1 mM and 2 mM magnesium were used. Also, using KLM2 instead of OLM2 does not impact primary sequencing metrics or secondary sequencing metrics.

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the examples provided herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A resynthesis kit comprising a thermophilic phosphatase and a polymerase.

2. The resynthesis kit according to claim 1, wherein the kit comprises the thermophilic phosphatase at a concentration of about 0.01 μM to about 1000 μM.

3. The resynthesis kit according to claim 1, wherein the thermophilic phosphatase is derived from a thermophile, wherein the thermophile is of the genus Pyrococcus.

4. The resynthesis kit according to claim 1, wherein the thermophilic phosphatase comprises an amino acid sequence as defined in SEQ ID NO: 1, or a functional variant or functional fragment thereof.

5. The resynthesis kit according to claim 1, wherein the thermophilic phosphatase has a denaturation temperature of above 40° C.

6. The resynthesis kit according to claim 1, wherein the kit comprises the polymerase at a concentration of about 0.01 μM to about 1000 μM.

7. The resynthesis kit according to claim 1, wherein the polymerase is a thermophilic polymerase.

8. The resynthesis kit according to claim 7, wherein the thermophilic polymerase has a denaturation temperature of above 40° C.

9. The resynthesis kit according to claim 1, wherein the kit further comprises a thermophilic glycosylase.

10. The resynthesis kit according to claim 9, wherein the kit comprises the thermophilic glycosylase at a concentration of about 0.01 μM to about 1000 μM.

11. The resynthesis kit according to claim 9, wherein the thermophilic glycosylase is derived from a thermophile, wherein the thermophile is of the genus Methanocaldococcus (Methanococcus).

12. The resynthesis kit according to claim 9, wherein the thermophilic glycosylase comprises an amino acid sequence as defined in SEQ ID NO: 2, or a functional variant or functional fragment thereof.

13. The resynthesis kit according to claim 9, wherein the thermophilic glycosylase has a denaturation temperature of above 40° C.

14. The resynthesis kit according to claim 1, wherein the kit further comprises a thermophilic exonuclease.

15. The resynthesis kit according to claim 14, wherein the kit comprises the thermophilic exonuclease at a concentration of between about 1 ug/ml and about 100 ug/ml.

16. The resynthesis kit according to claim 14, wherein the thermophilic exonuclease is derived from Pyrococcus abyssi or Pyrococcus furiosus.

17. The resynthesis kit of claim 16, wherein the thermophilic exonuclease comprises an amino acid sequence as defined in SEQ ID NO: 12, or a functional variant or functional fragment thereof.

18. The resynthesis kit of claim 16, wherein the thermophilic exonuclease comprises an amino acid sequence as defined in SEQ ID NO: 13, or a functional variant or functional fragment thereof.

19. The resynthesis kit of claim 1, wherein the thermophilic phosphatase is in a lyophilized formulation.

20. The resynthesis kit of claim 19, wherein the lyophilized formulation comprises the thermophilic phosphatase, a salt, an exonuclease, a detergent, and any one or more of magnesium chloride, acetate, or sulfate.

21. The resynthesis kit of claim 1, wherein the thermophilic phosphatase comprises Pyrococcus abyssi alkaline phosphatase.

22. The resynthesis kit of claim 9, wherein the thermophilic glycosylase is in a lyophilized formulation.

23. The resynthesis kit of claim 22, wherein the lyophilized formulation comprises the thermophilic glycosylase, a salt, and trehalose.

24. The resynthesis kit according to claim 1, wherein the kit comprises at least one selected from the group consisting of: a recombinase, a single-stranded nucleotide binding protein, nucleotide triphosphates (NTPs), an ATP-generating substrate and an ATP-generating enzyme.

25. (canceled)

26. (canceled)

27. The resynthesis kit according to claim 1, wherein the kit further comprises instructions for use of the kit in resynthesis of a nucleic acid template, or pairwise sequencing of a nucleic acid template.

28. Use of a resynthesis kit according to claim 1, in resynthesis of a nucleic acid template, or pairwise sequencing of a nucleic acid sequence.

29. A method of conducting resynthesis of a nucleic acid sequence, wherein the method comprises:
removing a blocking phosphate group from a blocked primer using a thermophilic phosphatase to form a deblocked primer; and
forming a second nucleic acid template extending from the deblocked primer using a polymerase and a first nucleic acid template.

30.-40. (canceled)
